(19) United States
(12) Patent Application Publication
(10) Pub. No.: US 2017/0209348 A1
(43) Pub. Date: Jul. 27, 2017

(54) BIOPHOTONIC COMPOSITIONS COMPRISING HALOGEN AND USES THEREOF

(71) Applicant: KLOX Technologies Inc., Laval (CA)

(72) Inventors: Remigio Piergallini, Grottammare (IT); Nikolaos Loupis, Athens (GR)

(21) Appl. No.: 15/321,713

(22) PCT Filed: Jun. 23, 2015
(86) PCT No.: PCT/CA2015/000407

§ 371 Date: Dec. 22, 2016

Related U.S. Application Data
(60) Provisional application No. 62/100,846, filed on Jan. 7, 2015.
(60) Provisional application No. 62/016,522, filed on Jun. 24, 2014.

(51) Int. Cl. A61K 8/20; A61N 5/06; A61K 8/49; A61K 41/00; A61Q 19/08

(52) U.S. Cl. CPC A61K 8/20; A61K 41/0057; A61Q 19/08; A61K 8/498; A61N 5/062; A61K 2800/81; A61N 2005/0662; A61N 2005/0659; A61N 2005/0661

(57) ABSTRACT

The present disclosure provides biophotonic compositions containing halogen ions and/or halogen salts and methods useful in phototherapy. In particular, the biophotonic compositions comprising halogen of the present disclosure include at least one chromophore; and halogens and/or halogen salts such KI, or KCl, or KBr, or CsBr, or MgBr₂, or ZnBr₂, or NaF, or NaCl, or NaBr, or I₂, or I₃⁻, or Br₂, or Cl₂. The biophotonic compositions and the methods of the present disclosure are useful for promoting tissue repair, wound healing, bone regeneration and skin rejuvenation, as well as treating oral diseases, microbial and viral infections, acne and various other skin disorders and various orphan diseases.

### FIELD OF THE DISCLOSURE

The present disclosure generally relates to biophotonic compositions comprising halogen for phototherapy.

### BACKGROUND OF THE DISCLOSURE

Phototherapy is recognized as having wide range of applications in both the medical and cosmetic fields. For example, phototherapy has been used to disinfect target sites as an antimicrobial treatment, to promote wound healing, and for skin rejuvenation.

One type of phototherapy comprises the topical application to a target tissue of compositions comprising chromophores. When activated by an incident light, the chromophores absorb and emit light such as through fluorescence with a therapeutic effect on its own and/or in combination with the incident light also irradiating the target tissue. Furthermore, the light activated chromophore may react with an oxygen source to generate oxygen radicals such as singlet oxygen which at low levels may also have a therapeutic effect on the target tissue.

However, in a process known as photobleaching, the chromophore may be degraded over time such as through attack by generated singlet oxygen. Such photobleaching of the chromophore in the composition may, however, may be undesirable since providing a longer time over which the chromophore may be irradiated and therefore emit fluorescence having a therapeutic effect and, as well, having a longer period over which the illuminated chromophore may interact with a source of oxygen, may provide for a therapeutic effect on a target tissue.

It is the object of the present disclosure to provide improved biophotonic compositions and methods useful in phototherapy.

### SUMMARY OF THE DISCLOSURE

The present disclosure provides, in some aspects, a method for extending the fluorescence lifespan of one or more chromophores comprising contacting the one or more chromophores with one or more halogens and/or halogen salts and exposing the resulting composition to actinic light. In some instances, the methods further comprise contacting the resulting composition with a peroxide or peroxide precursor. The present disclosure also provides the use of one or more halogens and/or halogen salts in combination with one or more chromophores to increase the fluorescence of the one or more chromophores and/or to increase time to photobleaching of the one or more chromophores.

The present disclosure also provides, in some aspects, biophotonic compositions comprising halogens and methods useful in phototherapy. In particular, a biophotonic composition of the present disclosure may include at least one chromophore; and halogens and/or halogen salts.

The present disclosure also provides, in some aspects, biophotonic compositions comprising one or more halogens and/or halogen salts and methods useful in phototherapy. In particular, a biophotonic composition of the present disclosure may include at least one chromophore; halogens and/or halogen salts; an oxidant, such as a peroxide or a peroxide precursor, and a carrier.

In one aspect, there is provided a biophotonic composition comprising: at least one chromophore; KI, or KCl, or KBr, or CsBr, or MgBr₂, or ZnBr₂, or NaF, or NaCl, or NaBr, or I₂, or I₃⁻, or Br₂, or Cl₂, or a combination thereof; an oxidant, such as a peroxide or a peroxide precursor, and a carrier. In some embodiments the biophotonic composition comprises KI. KI may be present in the biophotonic composition at a concentration of about 0.5 to 20 ppm, or about 1 to 10 ppm, or about 10 to 3,000 ppm, or about 50 to 2000 ppm, or about 100 to 1500 ppm, or about 100 to 1000 ppm, or about 100 to 500 ppm, or about 100 to 300 ppm, or about 200 ppm.

In certain embodiments of the foregoing or following, the oxidant is a peroxide or peroxide precursor. In some embodiments, the peroxide or peroxide precursor is selected from hydrogen peroxide, carbamide peroxide, benzoyl peroxide, peroxy acid, alkali metal peroxide, alkali metal percarbonate, peroxyacetic acid, alkali metal perborate, or methyl ethyl ketone peroxide. In some embodiments the peroxide is carbamide peroxide. The peroxide or peroxide precursor may be present in the biophotonic composition in an amount of about 0.01% to about 50% by weight of the final composition, or about 0.01% to about 5%, or about 1% to about 10%, or about 1% to about 20%.

In certain embodiments of the foregoing or following, the carrier comprises at least one of a hydrophilic agent, a hygroscopic agent or a hydrated polymer. In some embodiments the carrier is polyanionic in charge character. In further embodiments the carrier comprises carboxylic functional groups. In some embodiments the carrier comprises a polymer having from 2 to 7 carbon atoms per functional group.

In certain embodiments of the foregoing, or following the carrier comprises a synthetic polymer selected from vinyl polymers, poly(ethylene oxide), acrylamide polymers, polyoxyethylene-polyoxypropylene copolymers, and derivatives or salts thereof. In further embodiments the carrier is a vinyl polymer selected from polyacrylic acid, polymethacrylic acid, polyvinyl pyrrolidone or polyvinyl alcohol. The carrier may comprise a carboxy vinyl polymer or a carbomer obtained by polymerization of acrylic acid. The carboxy vinyl polymer or carbomer may be crosslinked. In some embodiments the carrier comprises Carbopol® 940 (e.g., a carbomer), Carbopol® 980, ETD 2020 NF, Carbopol® 1382 Polymer (Acrylates/C10-30 Alkyl Acrylate Crosspolymer), 71G NF, 971P NF, 974P NF, 980 NF, 981 NF, 5984 EP, ETF 2020 NF, ultrez 10 NF, ultrez 20, ultrez 21, 1342 NF, 934 NF, 934P NF, 940 NF, or 941 NF. In some embodiments the carrier comprises a polyacrylic acid polymer cross-linked with alkyl acrylate or allyl pentaerythritol and is present in an amount of about 0.05% to about 5% by weight of the final composition, or about 0.5% to about 2% by weight of the final composition.

In certain embodiments of the foregoing or following, the carrier comprises a protein-based polymer. In some embodiments the protein-based polymer is selected from at least one of sodium hyaluronate, gelatin and collagen. In some embodiments the carrier is gelatin and may be present in an amount of equal to or more than about 4% by weight of the final composition. In other embodiments the carrier is collagen and may be present in an amount equal to or more than about 5%, about 6%, about 7%, about 8%, about 9%, about 10% about 15%, about 20%, about 25%, or about 30% by weight of the final composition.

In certain embodiments of the foregoing or following, the carrier comprises a polysaccharide. In some embodiments the polysaccharide is selected from at least one of starch, chitosan, chitin, agar, alginates, xanthan, carrageenan, guar gum, gellan gum, pectin, and locust bean gum.

In some embodiments the carrier comprises at least one glycol. In further embodiments the glycol is selected from ethylene glycol and propylene glycol.

In certain embodiments of the foregoing or following, the carrier is a pharmaceutically acceptable carrier.

In certain embodiments, the carrier is water, saline, buffered saline or the like.

In certain embodiments of the foregoing or following, the at least one chromophore in the biophotonic composition is a fluorescent chromophore (fluorophore). In some embodiments the at least one chromophore absorbs and/or emits light within the visible range. In some embodiments, the chromophore is a synthetic chromophore. By “synthetic chromophore” is meant a chromophore that is synthesised artificially by man. In some embodiments, the chromophore is a natural chromophore. By “natural chromophore” is meant a chromophore that exists in nature and/or that is caused by nature. The natural chromophore may be isolated and/or purified from its naturally occurring environment/source. In some implementations of this embodiment, the natural chromophore is derived from a plant source, or, a fungal source or, an algal source or, a marine or terrestrial microorganism source or, an animal source.

In some aspects, a natural chromophore that is isolated and/or purified from its naturally occurring environment is in a form that is “purified”, “isolated” or “substantially pure”. The natural chromophores are “purified”, “isolated” or “substantially pure” when they are separated from the components that naturally accompany them. Typically, a compound is substantially pure when it is at least 60%, 65%, 70%, 75%, 80%, 85%, 90%, 95%, 96%, 97%, 98%, or 99%, by weight, of the total material in a sample.

In some embodiments the at least one chromophore absorbs and/or emits light within the range of about 400 nm to about 800 nm. The chromophore may absorb and/or emit light within the green, orange and yellow portions of the electromagnetic spectrum. In certain embodiments the at least one chromophore is a xanthene dye. In some embodiments, the xanthene dye is selected from Eosin Y, Eosin B, Erythrosin B, Fluorescein, Rose Bengal and Phloxin B. In some embodiments the chromophore is present in an amount of about 0.0001% to about 40%, or about 0.0001% to about 35%, or about 0.0001% to about 30%, or about 0.0001% to about 25%, or about 0.0001% to about 20%, or about 0.0001% to about 15%, or about 0.0001% to about 10%, or about 0.0001% to about 9%, or about 0.0001% to about 8%, or about 0.0001% to about 7%, or about 0.0001% to about 6%, or about 0.0001% to about 5%, or about 0.0001% to about 4%, or about 0.0001% to about 3%, or about 0.0001% to about 2% by weight of the total composition.

In some embodiments the biophotonic composition further comprises a second chromophore. In some embodiments, the first chromophore has an emission spectrum that overlaps with an absorption spectrum of the second chromophore. In some embodiments, the first chromophore has an emission spectrum that overlaps at least 20% with an absorption spectrum of the second chromophore. The first chromophore may transfer energy to the second chromophore upon illumination with light. In certain embodiments the first chromophore is Eosin Y and the second chromophore is one or more selected from Fluorescein, Phloxine B and Erythrosine B. In some embodiments the first chromophore is Eosin Y and the second chromophore is Fluorescein. The second chromophore may be present an amount of about 0.0001% to about 40%, or about 0.0001% to about 2% by weight of the total composition.

In further embodiments, the biophotonic composition comprises a third chromophore. The third chromophore may comprise chlorophyll or saffron. The third chromophore may be present in an amount of about 0.0001% to about 40%, or about 0.0001% to about 2% by weight of the total composition.

In certain embodiments of the foregoing or following, the biophotonic composition has a translucency of least about 40%, about 45%, about 50%, about 55%, about 60%, about 65%, about 70%, about 75%, about 80%, about 85%, about 90%, about 95% or about 100% in a visible range when measured without the chromophore present.

In certain embodiments or the foregoing or the following, the biophotonic composition is applied to substrates. As used herein the term “substrate” refers to a material onto which the biophotonic composition is applied. As used herein, the expression “treated substrate” refers to a substrate that has the biophotonic composition applied thereto. The substrate may be of fibrous nature, where fibers, either woven or non-woven form the interstices. Alternatively the substrate may be non-fibrous, such a synthetic foam (such as, for example, a sponge). Examples of a substrate include, but are not limited to, fibrous textiles including natural fibers either vegetal (such as cotton, linen, jute) or animal (such as wool and silk) as well as mineral fibers (such as asbestos and viscose); chemical fibers either synthetic or artificial like polyester, nylon, acetate, polypropolene and rayon; paper and paper products; product made from composites; products made from wood or wood byproducts, such as furniture materials and doors; products made from carbon fiber, products made from glass fiber, synthetic foam, such as polyethylene, polystyrene and polyurethane foam. Textiles may be woven, knitted or machine-knitted, or be present as a composite material (non-woven textile). In the case of composite materials, the fabric is not produced by wrap and weft or stitch formation, but by interlocking and/or cohesive and/or adhesive bonding of textile fibers. Non-woven fabrics are loose materials produced from spun fibers or filaments, in most cases made of polypropylene, polyester or viscose, the cohesion of which is generally provided by the fibers intrinsically holding together. In this regard, the individual fibers may have a preferred orientation (oriented or cross-laid non-woven fabrics), or be un-oriented (entangled non-woven fabrics). The non-woven fabrics may be mechanically bonded by needle punching, stitching, or entangling by means of strong water jets. Adhesively bonded non-woven fabrics are produced by gluing the fibers together with liquid binding agents (for example, acrylate polymers, SBR/NBR, polyvinyl ester, polyurethane dispersions), or by melting or dissolving so-called binder fibers that are added to the non-woven fabric during its production. Non-woven material may be obtained from, for example, viscose, cotton, cellulose, jute, hemp, sisal, silk, wool, polypropylene, polyester, polyethylene terephthalate (PET), aramide, nylon, polyvinyl derivatives, polyurethanes, polylactide, polyhydroxyalkanoate, cellulose esters and/or polyethylene, and also mineral fibers, such as glass fibers or carbon fibers. Examples of fabrics also include blends of dual or multiple fibers such as, but not limited to, polyester/elastane blends, polyamids, polyamide/elastane blends, cotton/polyester/elastane blends, polyacrylonitriles, acetates, modal, lyocell and linens.

In certain embodiments of the foregoing or following, the biophotonic composition is used for cosmetic or medical treatment of a tissue (such as a skin tissue). In some embodiments the cosmetic treatment includes skin rejuvenation and conditioning, and medical treatment includes wound healing, bone injury or disease repair, periodontitis other oral diseases treatment, and treatment of skin conditions. The skin condition may be acne, eczema, psoriasis or dermatitis. In some embodiments the biophotonic composition is used for modulating inflammation. In some embodiments the biophotonic composition is used for modulating collagen production. In other embodiments the biophotonic composition is used for promoting angiogenesis. In some embodiments the biophotonic composition is used for loosing or removing dry or dead skin. In some embodiments the biophotonic composition is used for treating bacterial, viral or fungal infections. In some embodiments the biophotonic composition is used for debridement of wounds or skin. In some embodiments, the medical treatment includes tissue repair, wound healing, oral disease treatment, periodontitis treatment, treatment of bacterial, viral or fungal infections, treatment of a fistula, treatment of skin conditions, or treatment of an orphan disease.

In another aspect, there is provided a method for biophotonic treatment of a skin disorder, wherein the method comprises applying a biophotonic composition to a target tissue (such as a skin tissue), wherein the biophotonic composition comprises at least one chromophore; and halogens and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof); and illuminating said biophotonic composition with light having a wavelength that overlaps with an absorption spectrum of the chromophore. In some implementations of this aspect, the biophotonic composition further comprises an oxidant, such as a peroxide or a peroxide precursor. The skin disorder may be acne, eczema, psoriasis or dermatitis.

In some implementations of this aspect, the light that may be useful for illumination of the biophotonic composition as defined herein is a continuous light. In some other implementations, the light that may be useful for illumination of the biophotonic composition as defined herein is a modulated light such as a pulsed light. In some implementations of this aspect, the light source that may be useful for illumination of the biophotonic composition as defined herein is a light-emitting diode (LED).

From a further aspect, there is provided a method for biophotonic treatment of acne, wherein the method comprises applying a biophotonic composition to a target skin tissue, wherein the biophotonic composition comprises at least one chromophore; halogens and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof); and illuminating said biophotonic composition with light having a wavelength that overlaps with an absorption spectrum of the chromophore. In some implementations of this aspect, the biophotonic composition further comprises an oxidant, such as peroxide or a peroxide precursor.

From another aspect, there is provided a method for promoting wound healing, wherein the method comprises applying a biophotonic composition to a target tissue (such as a wound), wherein the biophotonic composition comprises at least one chromophore; and halogens and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof); and illuminating said biophotonic composition with light having a wavelength that overlaps with an absorption spectrum of the chromophore. In some implementations of this aspect, the biophotonic composition further comprises a peroxide or a peroxide precursor.

From another aspect, there is provided a method for promoting skin rejuvenation, wherein the method comprises applying a biophotonic composition to a target skin tissue, wherein the biophotonic composition comprises at least one chromophore; and halogens and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof; and illuminating said biophotonic composition with light having a wavelength that overlaps with an absorption spectrum of the chromophore. In some implementations of this aspect, the biophotonic composition further comprises a peroxide or a peroxide precursor.

From another aspect, there is provided a kit comprising a first component comprising at least one chromophore; and a second component comprising halogen and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof).

From another aspect, there is provided a kit comprising a first component comprising at least one chromophore; a second component comprising halogen and/or halogen salts (such as KI, or KCl, or KBr, or CsBr, or MgBr₂, or ZnBr₂, or NaF, or NaCl, or NaBr, or I₂, or I₃⁻, or Br₂, or Cl₂, or a combination thereof); a third component comprising a peroxide or a peroxide precursor, and a fourth component comprising a carrier, wherein one or more of the components may come in separate containers within the kit.

Instructions for use may be provided with the kit in some embodiments. In another embodiment the kit may comprise the biophotonic compositions of any of the foregoing claims and a light source for activating the at least one chromophore. The light source may be a lamp such as an LED lamp.

### BRIEF DESCRIPTION OF THE DRAWINGS

Further aspects and advantages of the present invention will become better understood with reference to the description in association with the following in which:

FIGS. 1 and 2 illustrate the peak fluorescence emission of Eosin Y in aqueous solution with carbamide peroxide (CP), with (FIG. 1) and without KI (FIG. 2), when activated by a blue light, according to certain aspects of the present disclosure. It can be seen that the KI changes the photobleaching profile over time of the Eosin Y.

FIG. 3 shows FIGS. 1 and 2 overlaid.

FIGS. 4 and 5 show the peak fluorescence emission of Eosin Y in carbomer gel with carbamide peroxide, with (FIG. 4) and without KI (FIG. 5), when activated by a blue light, according to certain aspects of the present disclosure.

FIG. 6 is a graph showing the curves of FIGS. 4 and 5 overlaid, together with the photobleaching curves of Eosin Y in a carbomer gel with and without KI in the absence of carbamide peroxide.

FIG. 7 illustrates the effect of different concentrations of KI on the amount of fluorescence emitted by Eosin Y in the presence of peroxide, according to certain aspects of the present disclosure.

FIG. 8 shows the light spectrum recorded during exposure of a biophotonic composition comprising 109 ug/g Eosin Y and 12% urea peroxide to blue light for 5 minutes.

FIG. 9 shows the light spectrum recorded during exposure of a biophotonic composition comprising 109 ug/g Eosin Y, 12% urea peroxide and 50 ppm KI to blue light for 5 minutes.

FIG. 10 shows the light spectrum recorded during exposure of a biophotonic composition comprising 109 ug/g Eosin Y, 12% urea peroxide and 200 ppm KI to blue light for 5 minutes.

FIG. 11 shows the light spectrum recorded during exposure of a biophotonic composition comprising 109 ug/g Eosin Y, 12% urea peroxide and 500 ppm KI to blue light for 5 minutes.

FIG. 12 shows the light spectrum recorded during exposure of a biophotonic composition comprising 109 ug/g Eosin Y, 12% urea peroxide and 1000 ppm KI to blue light for 5 minutes.

FIG. 13 shows the light spectrum recorded during exposure of a biophotonic composition comprising 109 ug/g Eosin Y, 12% urea peroxide and 3000 ppm KI to blue light for 5 minutes.

FIG. 14 shows the light spectrum recorded during exposure of a biophotonic composition comprising 109 ug/g Eosin Y, 12% urea peroxide and 5000 ppm KI to blue light for 5 minutes.

FIG. 15 is a photograph of carbomer gel comprising 109 ug/g Eosin Y, 12% urea peroxide and 5000 ppm KI. Side A of the gel was illuminated with blue light for 5 minutes, while side B was not illuminated with blue light.

FIG. 16 is a photograph of the carbomer gel of FIG. 15 following a 5 minute illumination of one half of the gel with blue light.

### DETAILED DESCRIPTION

### (1) Overview

The present disclosure provides biophotonic compositions comprising halogen and uses thereof. Illumination of halogen containing biophotonic compositions results in increased time to photobleaching of the chromophore compared to halogen-free compositions. Consequently, the chromophore is available for longer periods of time and can thus interact with a source of oxygen to produce reactive oxygen species and/or to fluoresce and emit increased fluorescence. In certain embodiments, phototherapy using the halogen containing biophotonic compositions of the present disclosure may promote rejuvenation of the skin, promote wound healing, treat skin conditions such as acne, eczema and dermatitis, loosen or remove dry or dead skin, debride wounds, or treat periodontitis.

### (2) Definitions

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions exert their physiological effects primarily due to the generation and manipulation of photons, for example, by absorbing photon to emit photons or to transfer energy, for example, by absorbing photons to emit photons or to transfer energy.

Terms “chromophore” and “photoactivator” are used herein interchangeably. A chromophore means a chemical compound, when contacted by light irradiation, is capable of absorbing the light. The chromophore readily undergoes photoexcitation and can transfer its energy to other molecules or emit it as light (e.g. fluorescence).

“Photobleaching” or “photobleaches” means the photochemical destruction of a chromophore. A chromophore may fully or partially photobleach.

The term “actinic light” is intended to mean light energy emitted from a specific light source (e.g. lamp, LED, laser or sunlight) and capable of being absorbed by matter (e.g. the chromophore or photoactivator). The expression “actinic light” and the term “light” are used herein interchangeably. In some embodiments, the actinic light is visible light.

“Topical application” or “topical uses” means application to body surfaces, such as the skin, mucous membranes, vagina, oral cavity, internal surgical wound sites, and the like.

“Skin rejuvenation” means a process of reducing, diminishing, retarding or reversing one or more signs of skin aging or generally improving the condition of skin. For instance, skin rejuvenation may include increasing luminosity of the skin, reducing pore size, reducing fine lines or wrinkles, improving thin and transparent skin, improving firmness, improving sagging skin (such as that produced by bone loss), improving dry skin (which might itch), reducing or reversing freckles, reducing or preventing the appearance of age spots, spider veins, rough and leathery skin, fine wrinkles that disappear when stretched, reducing loose skin, or improving a blotchy complexion. According to the present disclosure, one or more of the above conditions may be improved or one or more signs of aging may be reduced, diminished, retarded or even reversed by certain embodiments of the compositions, methods and uses of the present disclosure.

“Wound” means an injury to any tissue, including for example, acute, subacute, delayed or difficult to heal wounds, and chronic wounds. Examples of wounds may include both open and closed wounds. Wounds include, for example, amputations, burns, incisions, excisions, lesions, lacerations, abrasions, puncture or penetrating wounds, surgical wounds, amputations, contusions, hematomas, crushing injuries, ulcers (such as for example pressure, diabetic, venous or arterial), scarring (cosmesis), wounds caused by periodontitis (inflammation of the periodontium).

Features and advantages of the subject matter hereof will become more apparent in light of the following detailed description of selected embodiments. As will be realized, the subject matter disclosed and claimed is capable of modifications in various respects, all without departing from the scope of the claims. Accordingly, the examples and the description are to be regarded as illustrative in nature, and not as restrictive and the full scope of the subject matter is set forth in the claims.

### (3) Biophotonic Compositions Comprising Halogen

The present disclosure provides, in a broad sense, biophotonic compositions comprising halogen which can be activated by light (e.g., photons) of specific wavelengths. A biophotonic composition according to various embodiments of the present disclosure contains at least one chromophore; halogens and/or halogen salts; an oxidant, such as a peroxide or a peroxide precursor, and a carrier. Illumination of the biophotonic composition may lead to the generation of oxygen radicals such as singlet oxygen, and fluorescence, which individually or together may have a therapeutic effect.

When a chromophore absorbs a photon of a certain wavelength, it becomes excited. This is an unstable condition and the molecule tries to return to the ground state, giving away the excess energy. For some chromophores, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift. In the proper environment (e.g., in a biophotonic composition) much of this energy is transferred to the other components of the biophotonic composition or to the treatment site directly.

Without being bound to theory, it is thought that fluorescence emitted by photoactivated chromophores may have therapeutic properties due to its femto-, pico-, or nano-second emission properties which may be recognized by biological cells and tissues, leading to favorable biomodulation. Furthermore, the emitted fluorescence has a longer wavelength and hence penetrates deeper into tissue. Irradiating tissue with such a broad range of wavelength, including in some embodiments the activating light which passes through the composition, may have different and complementary effects on the cells and tissues. In other words, chromophores are used in the biophotonic compositions of the present disclosure for therapeutic effect on tissues. This is a distinct application of these photoactive agents and differs from the use of chromophores as simple stains or as catalysts for photo-polymerization.

The biophotonic compositions of the present disclosure may be described based on the components making up the composition. Additionally or alternatively, the compositions of the present disclosure have functional and structural properties and these properties may also be used to define and describe the compositions. Individual components of the biophotonic compositions of the present disclosure, including chromophores, halogens and/or halogen salts, oxidants (peroxides and peroxide precursors), carriers and other optional ingredients, are detailed below.

(a) Chromophores

Suitable chromophores can be fluorescent compounds (or stains) (also known as “fluorochromes” or “fluorophores”). Other dye groups or dyes (biological and histological dyes, food colorings, carotenoids, and other dyes) can also be used. Suitable chromophores can be synthetic or can be natural. Some suitable natural chromophores are derived from a plant source. In some implementations, the plant-derived chromophore is obtained from a plant extract, for example, but not limited to, extracts of coffee beans, green tea leaves, blueberries, cranberries, huckleberries, acai berries, goji berries, blackberries, raspberries, grapes, strawberries, persimmon, pomegranate, lingonberry, bearberry, mulberry, bilberry, choke cherry, sea buckthorn berries, goji berry, tart cherry, kiwi, plum, apricot, apple, banana, berry, blackberry, blueberry, cherry, cranberry, currant, greengage, grape, grapefruit, gooseberry, lemon, mandarin, melon, orange, pear, peach, pineapple, plum, raspberry, strawberry, sweet cherry, watermelon, and wild strawberry. In some embodiments, the plant-derived chromophore is obtained from trees, including for instance sequoia, coastal redwood, bristlecone pine, birch, and cedar.

Suitable photoactivators can be those that are Generally Regarded As Safe (GRAS). Advantageously, photoactivators which are not well tolerated by the skin or other tissues can be included in the biophotonic composition of the present disclosure, as in certain embodiments, the photoactivators are encapsulated within a carrier and do not contact the tissues.

The biophotonic composition of the present disclosure comprises at least one chromophore. In some embodiments, the chromophore absorbs at a wavelength in the range of the visible spectrum, such as at a wavelength of about 380-800 nm, 380-700, 400-800, or 380-600 nm. In other embodiments, the chromophore absorbs at a wavelength of about 200-800 nm, 200-700 nm, 200-600 nm or 200-500 nm. In one embodiment, the chromophore absorbs at a wavelength of about 200-600 nm. In some embodiments, the chromophore absorbs light at a wavelength of about 200-300 nm, 250-350 nm, 300-400 nm, 350-450 nm, 400-500 nm, 450-650 nm, 600-700 nm, 650-750 nm or 700-800 nm.

It will be appreciated to those skilled in the art that optical properties of a particular chromophore may vary depending on the chromophore's surrounding medium.

Therefore, as used herein, a particular chromophore's absorption and/or emission wavelength (or spectrum) corresponds to the wavelengths (or spectrum) measured in a biophotonic compositions of the present disclosure.

The biophotonic composition disclosed herein may include at least one additional chromophore. Combining chromophores may increase photo-absorption by the combined dye molecules and enhance absorption and photo-biomodulation selectivity. Thus, in certain embodiments, biophotonic compositions of the disclosure include more than one chromophore. When such multi-chromophore materials are illuminated with light, energy transfer can occur between the chromophores. This process, known as resonance energy transfer, is a widely prevalent photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. More specifically, for energy transfer to occur, the emission spectrum of the donor chromophore must overlap with the absorption spectrum of the acceptor chromophore.

Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, the more overlap there is between the donor chromophore's emission spectra and the acceptor chromophore's absorption spectra, the better a donor chromophore can transfer energy to the acceptor chromophore.

In certain embodiments, the biophotonic composition of the present disclosure further comprises a second chromophore. In some embodiments, the first chromophore has an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the second chromophore. In one embodiment, the first chromophore has an emission spectrum that overlaps at least about 20% with an absorption spectrum of the second chromophore. In some embodiments, the first chromophore has an emission spectrum that overlaps at least between about 1-10%, between about 5-15%, between about 10-20%, between about 15-25%, between about 20-30%, between about 25-35%, between about 30-40%, between about 35-45%, between about 50-60%, between about 55-65% or between about 60-70% with an absorption spectrum of the second chromophore.

In certain embodiments, the biophotonic composition of the present disclosure further comprises a third chromophore. In some embodiments, the second chromophore has an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the third chromophore. In one embodiment, the second chromophore has an emission spectrum that overlaps at least about 20% with an absorption spectrum of the third chromophore. In some embodiments, the second chromophore has an emission spectrum that overlaps at least between about 1-10%, between about 5-15%, between about 10-20%, between about 15-25%, between about 20-30%, between about 25-35%, between about 30-40%, between about 35-45%, between about 50-60%, between about 55-65% or between about 60-70% with an absorption spectrum of the third chromophore.

% spectral overlap, as used herein, means the % overlap of a donor chromophore's emission wavelength range with an acceptor chromophore's absorption wavelength rage, measured at spectral full width quarter maximum (FWQM). For example, if the spectral FWQM of the acceptor chromophore's absorption spectrum is about 60 nm and the overlap of the donor chromophore's spectrum with the absorption spectrum of the acceptor chromophore is about 30 nm, then the % overlap can be calculated as 30 nm/60 nm×100=50%.

In some embodiments, the second chromophore absorbs at a wavelength in the range of the visible spectrum. In certain embodiments, the second chromophore has an absorption wavelength that is relatively longer than that of the first chromophore within the range of about 50-250 nm, 25-150 nm or 10-100 nm. In some embodiments, the third chromophore absorbs at a wavelength in the range of the visible spectrum. In certain embodiments, the third chromophore has an absorption wavelength that is relatively longer than that of the second chromophore within the range of about 50-250 nm, 25-150 nm or 10-100 nm.

The first chromophore can be present in an amount of about 0.001-40% per weight of the biophotonic composition. When present, the second chromophore can be present in an amount of about 0.001-40% per weight of the biophotonic composition. When present, the third chromophore can be present in an amount of about 0.001-40% per weight of the biophotonic composition. In certain embodiments, the first chromophore is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic composition. In certain embodiments, the second chromophore is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic composition. In certain embodiments, the third chromophore is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic composition. In certain embodiments, the total weight of chromophore or combination of chromophores may be in the amount of about 0.005-1%, 0.05-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40.001% per weight of the biophotonic composition.

The concentration of the chromophore to be used can be selected based on the desired intensity and duration of the biophotonic activity from the biophotonic composition, and on the desired medical or cosmetic effect. For example, some dyes such as xanthene dyes reach a ‘saturation concentration’ after which further increases in concentration do not provide substantially higher emitted fluorescence. Further increasing the chromophore concentration above the saturation concentration can reduce the amount of activating light passing through the matrix. Therefore, if more fluorescence is required for a certain application than activating light, a high concentration of chromophore can be used. However, if a balance is required between the emitted fluorescence and the activating light, a concentration close to or lower than the saturation concentration can be chosen.

Suitable chromophores that may be used in the biophotonic compositions of the present disclosure include, but are not limited to, the following:

Chlorophyll Dyes

Examples of chlorophyll dyes include but are not limited to chlorophyll a; chlorophyll b; chlorophyllin, oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Xanthene Derivatives

Examples of xanthene dyes include but are not limited to eosin B, eosin B (4′,5′-dibromo,2′,7′-dinitr-o-fluorescein, dianion); Eosin Y; eosin Y (2′,4′,5′,7-tetrabromo-fluoresc-ein, dianion); eosin (2′,4′,5′,7-tetrabromo-fluorescein, dianion); eosin (2′,4′,5′,7′-tetrabromo-fluorescein, dianion) methyl ester, eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester, 4,5-dibromo-rhodamine n-butyl ester, rhodamine 101 methyl ester, rhodamine 123; rhodamine 6G; rhodamine 6G hexyl ester; tetrabromo-rhodamine 123; and tetramethyl-rhodamine ethyl ester.

Methylene Blue Dyes

Examples of methylene blue derivatives include but are not limited to 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenothiazine.

Azo Dyes

Examples of azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more chromophores of the biophotonic compositions disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin Green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural green 3(chlorophyllin), Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, phycobilins, Phycocyanins, Phycoerythrins. Phycoerythrincyanin (PEC), Phthalocyanines, Pyronin G, Pyronin Y, Quinine, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Vitamin B, Water blue I, Water soluble eosin, Xylidine ponceau, and Yellowish eosin.

In certain embodiments, the biophotonic composition of the present disclosure includes any of the chromophores listed above, or a combination thereof, so as to provide a synergistic biophotonic effect at the application site.

Without being bound to any particular theory, a synergistic effect of the chromophore combinations means that the biophotonic effect is greater than the sum of their individual effects. Advantageously, this may translate to increased reactivity of the biophotonic composition, faster or improved treatment time. Also, the treatment conditions need not be altered to achieve the same or better treatment results, such as time of exposure to light, power of light source used, and wavelength of light used. In other words, use of synergistic combinations of chromophores may allow the same or better treatment without necessitating a longer time of exposure to a light source, a higher power light source or a light source with different wavelengths.

In some embodiments, the composition includes Eosin Y as a first chromophore and any one or more of Rose Bengal, Fluorescein, Erythrosine, Phloxine B, chlorophyllin as a second chromophore. It is believed that these combinations have a synergistic effect as they can transfer energy to one another when activated due in part to overlaps or close proximity of their absorption and emission spectra. This transferred energy is then emitted as fluorescence or leads to production of reactive oxygen species. This absorbed and re-emitted light is thought to be transmitted throughout the composition, and also to be transmitted into the site of treatment.

In further embodiments, the composition includes the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. Other synergistic chromophore combinations are also possible.

By means of synergistic effects of the chromophore combinations in the composition, chromophores which cannot normally be activated by an activating light (such as a blue light from an LED), can be activated through energy transfer from chromophores which are activated by the activating light. In this way, the different properties of photoactivated chromophores can be harnessed and tailored according to the cosmetic or the medical therapy required.

For example, Rose Bengal can generate a high yield of singlet oxygen when activated in the presence of molecular oxygen, however it has a low quantum yield in terms of emitted fluorescent light. Rose Bengal has a peak absorption around 540 nm and so can be activated by green light. Eosin Y has a high quantum yield and can be activated by blue light. By combining Rose Bengal with Eosin Y, one obtains a composition which can emit therapeutic fluorescent light and generate singlet oxygen when activated by blue light. In this case, the blue light photoactivates Eosin Y which transfers some of its energy to Rose Bengal as well as emitting some energy as fluorescence.

In embodiments of the biophotonic composition comprising a third chromophore, the third chromophore may be a chlorophyll or saffron. Saffron is a spice derived from crocus sativus. Saffron contains more than 150 different compounds many of them are carotenoids: mangicrocin, reaxanthine, lycopene, and various α- and β-carotenes, that show good absorption of light and beneficial biological activity. Also saffron can act as both a photon-transfer agent and a healing factor.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 10 mW/cm², about 0.5 to about 5 mW/cm².

(b) Halogens

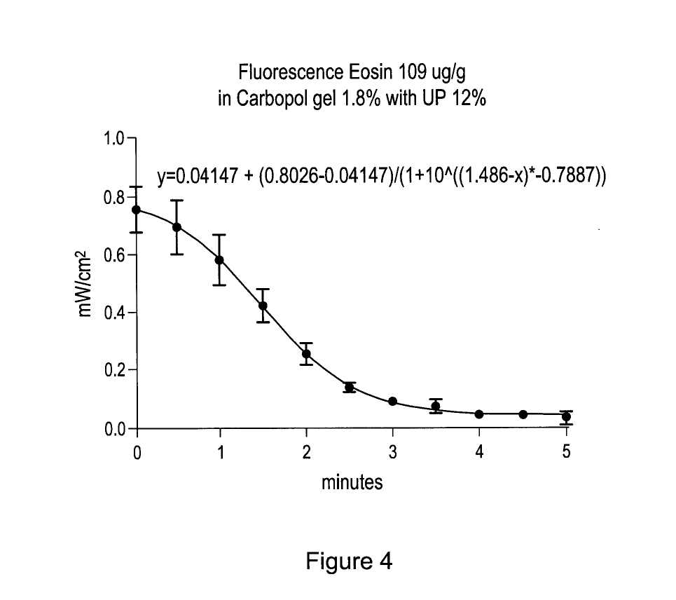

The biophotonic composition of the present disclosure includes halogens or halogen salts. It was found that the addition of halogens or halogen salts to the biophotonic composition significantly increased the amount of singlet oxygen produced on the illumination of the composition (See Tables 1 and 2). Furthermore, it was found that the maximum emitted fluorescence was increased and the time to photobleaching of the chromophore was extended (See FIGS. 4-6, FIG. 7, Table 3 and Table 4). Halogens are elements found in Group 17 of the periodic table and as such they have similar chemical properties such as high electronegativity.

In some embodiments of the present disclosure the composition comprises KI, or KCl, or KBr, or CsBr, or MgBr₂, or ZnBr₂, or NaCl, or NaBr, or I₂, or I₃⁻, or Br₂, or Cl₂, or other suitable salts or any combination thereof. These compounds will all generate halogen ions when dissolved in solution. Without being bound to theory, it is thought that chlorine, bromine and/or iodine ions are able to promote intersystem crossing in a chromophore. Intersystem crossing is the transition from a singlet excited state to a triplet excited state. It is thought that this phenomenon can generate more reactive oxygen species. Furthermore, we observe that addition of halogen ions increases the time to photobleaching of the chromophore. The total concentration of halogen and/or halogen salts that can be used in the biophotonic compositions is from about 1 to about 5,000 ppm, or about 10 to about 500 ppm, or about 50 to about 250 ppm.

In some embodiments the halogen salt is KI. A suitable concentration of KI that can be used in the biophotonic composition is from about 1 to about 5,000 ppm, or about 10 to about 500 ppm, or about 50 to about 250 ppm, or about 200 ppm.

In some implementations of these embodiments, the inclusion of the halogen in the biophotonic composition may create a favorable environment for the chromophores within the composition, thereby aiding the photoactive properties of the chromophore. In some instances, the halogen may promote the overall chromophore structural stability and prevent degradation of the chromophore. The halogen may also contribute to maximize the amount of oxygen generated and/or to prolong the generation of oxygen from the peroxide species.

(c) Oxidants

The biophotonic composition comprises a source of oxygen, such as at least one oxidant as a source of singlet oxygen or oxygen radicals. Peroxide compounds are oxidants that contain the peroxy group (R—O—O—R), which is a chainlike structure containing two oxygen atoms, each of which is bonded to the other and a radical or some element. When a biophotonic composition of the present disclosure comprising an oxidant is illuminated with light, the chromophores are excited to a higher energy state. When the chromophores' electrons return to a lower energy state, they emit photons with a lower energy level, thus causing the emission of light of a longer wavelength (Stokes' shift). In the proper environment, some of this energy is transferred to oxygen or the reactive hydrogen peroxide and causes the formation of oxygen radicals, such as singlet oxygen. The singlet oxygen and other reactive oxygen species generated by the activation of the biophotonic composition are thought to operate in a hormetic fashion. That is, a health beneficial effect that is brought about by the low exposure to a normally toxic stimuli (e.g. reactive oxygen), by stimulating and modulating stress response pathways in cells of the targeted tissues. Endogenous response to exogenous generated free radicals (reactive oxygen species) is modulated in increased defense capacity against the exogenous free radicals and induces acceleration of healing and regenerative processes. Furthermore, the extreme sensitivity of bacteria to exposure to free radicals makes the biophotonic composition of the present disclosure potentially a bactericidal composition.

Peroxide compounds are oxidants that contain the peroxy group (R—O—O—R), which is a chainlike structure containing two oxygen atoms, each of which is bonded to the other and a radical or some element. Suitable oxidants for preparation of the active medium include, but are not limited to:

Hydrogen peroxide (H₂O₂) is a powerful oxidizing agent, and breaks down into water and oxygen and does not form any persistent, toxic residual compound. A suitable range of concentration over which hydrogen peroxide can be used in the biophotonic composition is from about 0.01% to 30%, about 1 to 25%, about 5% to 20%, about 10 to 15%, or less than about 20%.

Urea hydrogen peroxide (also known as urea peroxide, carbamide peroxide or percarbamide) is soluble in water and contains approximately 35% hydrogen peroxide. Urea peroxide brakes down to urea and hydrogen peroxide in a slow-release fashion that can be accelerated with heat or photochemical reactions. The released urea [(NH₂)₂CO₂], is highly soluble in water and is a powerful protein denaturant. It increases solubility of some proteins and enhances rehydration of the skin and/or mucosa. A suitable range of concentration over which urea peroxide can be used in the biophotonic composition of the present disclosure is less than about 25%, or less than about 20%, or less than about 15%, or less than about 10%, or less than about 5%, or from 0.1 to 5%, or from about 1% to about 15%.

Benzoyl peroxide consists of two benzoyl groups (benzoic acid with the H of the carboxylic acid removed) joined by a peroxide group. It is found in treatments for acne, in concentrations varying from 2.5% to 10%. The released peroxide groups are effective at killing bacteria. Benzoyl peroxide also promotes skin turnover and clearing of pores, which further contributes to decreasing bacterial counts and reduce acne. Benzoyl peroxide breaks down to benzoic acid and oxygen upon contact with skin, neither of which is toxic. A suitable range of concentration over which benzoyl peroxide can be used in the biophotonic composition is from about 2.5% to about 20%, or about 2.5% to about 10%.

In some embodiments the peroxide or peroxide precursor is peroxy acid, alkali metal peroxide, alkali metal percarbonate, peroxyacetic acid, alkali metal perborate, or methyl ethyl ketone peroxide. In some embodiments the oxidant is methyl ethyl ketone peroxide. A suitable range of concentration over which methyl ethyl ketone peroxide can be used in the biophotonic composition is from 0.01% to 15%.

(d) Carrier

The biophotonic compositions of the present disclosure comprise a carrier. In some embodiments, the carrier comprises a thickening agent and is present in an amount and ratio sufficient to provide a desired viscosity, flexibility, rigidity, tensile strength, tear strength, elasticity, and adhesiveness. The thickening agents are selected so that the chromophore can remain photoactive in the composition. The thickening agents are also selected according to their optical transparency. The composition should be able to transmit sufficient light to activate the at least one chromophore and, in embodiments where fluorescence is emitted by the activated chromophore, the composition should also be able to transmit the emitted fluorescent light to tissues. For example, the inventors have noted that some xanthene dyes do not fluoresce in non-hydrated carriers, so hydrated polymers or polar solvents may advantageous. The thickening agents should also be selected according to the intended use. For example, if the biophotonic composition comprising halogen is to be applied onto tissue, the carrier is preferably biocompatible, or the carrier has an outside layer of a biocompatible composition which will interface the tissue.

Thickening Agents

In some embodiments, the thickening agent is present in the composition in an amount of from about 0.001% to about 40% (w/w %) of the total weight. In certain embodiments, the total content of the thickening agent is about 0.001-0.01%, about 0.005-0.05%, about 0.01-0.1, about 0.05-0.5% about 0.1-1%, about 0.5-5%, about 1-5%, about 2.5-7.5%, about 5-10%, about 7.5-12.5%, about 10-15%, about 12.5-17.5%, or about 15-20%, or about 15-25%, or about 20-30/%, or about 25-35%, or about 30-40%. It will be recognized by one of skill in the art that the viscosity, flexibility, rigidity, tensile strength, tear strength, elasticity, and adhesiveness can be adjusted by varying the content of the thickening agent. Methods of determining viscosity, flexibility, rigidity, tensile strength, tear strength, elasticity, and adhesiveness are known in the art.

Thickening agents that can be used include but are not limited to a hydrophilic agent, a hygroscopic agent or a hydrated polymer. The thickening agent may be polyanionic in charge character. The thickening agent may comprise carboxylic functional groups, and may further contain 2 to 7 carbon atoms per functional group. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, combinations thereof.

In certain embodiments the at least one thickening agent is a synthetic polymer selected from one or more of vinyl polymers, polyoxythylene-polyoxypropylene copolymers, poly(ethylene oxide), acrylamide polymers and derivatives and salts thereof. In a further embodiment the vinyl polymer is one or more of polyacrylic acid, polymethacrylic acid, polyvinyl pyrrolidone and polyvinyl alcohol. In other embodiments the vinyl polymer is a carboxy vinyl polymer or a carbomer obtained by the polymerization of acrylic acid. The carboxy vinyl polymer or carbomer may be cross-linked.

As mentioned above, in some embodiments, the at least one thickening agent is a carbomer. Carbomers are synthetic high molecular weight polymers of acrylic acid that are crosslinked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomers are available as fine white powders which disperse in water to form acidic colloidal suspensions (a 1% dispersion has approximately pH 3) of low viscosity. Neutralization of these suspensions using a base, for example sodium, potassium or ammonium hydroxides, low molecular weight amines and alkanolamines, results in the formation of clear translucent gels.

In some embodiments, the carbomer is a Carbopol®. Such polymers are commercially available from B.F. Goodrich or Lubrizol under the designation Carbopol® 71G NF, 420, 430, 475, 488, 493, 910, 934, 934P, 940, 971P NF, 974P NF, 980 NF, 981 NF and the like. Carbopols are versatile controlled-release polymers, as described by Brock (Pharmacotherapy, 14:430-7 (1994)) and Durrani (Pharmaceutical Res. (Supp.) 8:S-135 (1991)), and belong to a family of carbomers which are synthetic, high molecular weight, non-linear polymers of acrylic acid, cross-linked with polyalkenyl polyether. In certain embodiments, the carbomer is Carbopol® 940, Carbopol® 980, ETD 2020NF, Carbopol® 1382, 71G NF, 971P NF, 974P NF, 980 NF, 981 NF, 5984 EP, ETF 2020 NF, Ultrez 10 NF, Ultrez 20, Ultrez 21, 1342 NF, 934 NF, 934P NF, 940 NF or 941 NF. In some embodiments, the carbomer is cross-linked with alkyl acrylate or allyl pentaerythritol. In some embodiments, the carbomer is present in the composition in an amount of from about 0.01 wt % to 15 wt %, or about 0.05 wt % to about 5 wt %, or about 0.5 wt % to about 2 wt %.

In certain embodiments the at least one thickening agent is a glycol, such as ethylene glycol or propylene glycol. In further embodiments, the at least one thickening agent is a poly (ethylene oxide) polymer (such as POLYOX™ from Dow Chemical), linear PVP and cross-linked PVP, PEG/PPG copolymers (such as BASF Pluracare® L1220), ethylene oxide (EO)-propylene oxide (PO) block copolymers (such as polymers sold under the trade mark Pluronic available from BASF Corporation), ester gum, shellac, pressure sensitive silicone adhesives (such as Bio-PSA from Dow-Corning), or mixtures thereof. In some embodiments, a copolymer comprises (PVM/MA). In an embodiment, a copolymer comprises poly (methylvinylether/maleic anhydride). In some embodiments, a copolymer comprises poly (methylvinylether/maleic acid). In some embodiments, a copolymer comprises poly (methylvinylether/maleic acid) half esters. In some embodiments, a copolymer comprises poly (methylvinylether/maleic acid) mixed salts.

In certain embodiments of the disclosure, the at least one thickening agent is a protein-based polymer. Such protein-based polymer may be selected from at least one of sodium hyaluronate, gelatin and collagen. For example, the composition may comprise at least about 4 wt %, about 4 wt % to about 25 wt %, or about 10 wt % to about 20 wt % gelatin within the biophotonic composition. The composition may comprise at least about 5 wt %, about 5 wt % to about 25 wt %, or about 10 wt % to about 20 wt % collagen and/or sodium hyaluronate within the biophotonic composition. Alternatively, a lower weight percentage of protein-based polymers may be used together with chemical cross-linkers or any other cross-linking means.

In certain embodiments of the disclosure, the at least one thickening agent is a polysaccharide, which may be selected from starch, chitosan, chitin, agar, alginates, xanthan, carrageenan, guar gum, gellan gum, pectin, and locust bean gum.

The biophotonic composition of the present disclosure may optionally be provided with a water-insoluble substrate. By “water insoluble”, it is meant that the substrate does not dissolve in or readily break apart upon immersion in water. In some embodiments, the water-insoluble substrate is the implement or vehicle for delivering the treatment composition to the skin or target tissue. A wide variety of materials can be used as the water-insoluble substrate. One or more of the non-limiting characteristics may be desirable: (i) sufficient wet strength for use, (ii) sufficient softness, (iii) sufficient thickness, (iv) appropriate size, (v) air permeability, and (vi) hydrophilicity.

Non-limiting examples of suitable water-insoluble substrates which meet the above criteria include nonwoven substrates, woven substrates, hydroentangled substrates, air entangled substrates, natural sponges, synthetic sponges, polymeric netted meshes, and the like. Preferred embodiments employ nonwoven substrates since they are economical and readily available in a variety of materials. By “nonwoven”, it is meant that the layer is comprised of fibers which are not woven into a fabric but rather are formed into a sheet, mat, or pad layer.

(e) Antimicrobials

Antimicrobials kill microbes or inhibit their growth or accumulation, and are optionally included in the biophotonic composition of the present disclosure. Suitable antimicrobials for use in the methods and compositions of the present disclosure include, but not limited to, phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

Additionally, the biophotonic composition of the present disclosure comprises a peroxide or peroxide derivative, which upon illumination of the biophotonic composition will lead to the generation oxygen radicals. The extreme sensitivity of bacteria to exposure to free radicals makes the biophotonic composition of the present disclosure potentially a bactericidal composition.

Examples of phenolic and chlorinated phenolic antimicrobial agents that can be used in the disclosure include, but are not limited to: phenol; 2-methyl phenol; 3-methyl phenol; 4-methyl phenol; 4-ethyl phenol; 2,4-dimethyl phenol; 2,5-dimethyl phenol; 3,4-dimethyl phenol; 2,6-dimethyl phenol; 4-n-propyl phenol; 4-n-butyl phenol; 4-n-amyl phenol; 4-tert-amyl phenol; 4-n-hexyl phenol; 4-n-heptyl phenol; mono- and poly-alkyl and aromatic halophenols; p-chlorophenyl; methyl p-chlorophenol; ethyl p-chlorophenol; n-propyl p-chlorophenol; n-butyl p-chlorophenol; n-amyl p-chlorophenol; sec-amyl p-chlorophenol; n-hexyl p-chlorophenol; cyclohexyl p-chlorophenol; n-heptyl p-chlorophenol; n-octyl; p-chlorophenol; o-chlorophenol; methyl o-chlorophenol; ethyl o-chlorophenol; n-propyl o-chlorophenol; n-butyl o-chlorophenol; n-amyl o-chlorophenol; tert-amyl o-chlorophenol; n-hexyl o-chlorophenol; n-heptyl o-chlorophenol; o-benzyl p-chlorophenol; o-benxyl-m-methyl p-chlorophenol; o-benzyl-m,m-dimethyl p-chlorophenol; o-phenylethyl p-chlorophenol; o-phenylethyl-m-methyl p-chlorophenol; 3-methyl p-chlorophenol 3,5-dimethyl p-chlorophenol, 6-ethyl-3-methyl p-chlorophenol, 6-n-propyl-3-methyl p-chlorophenol; 6-iso-propyl-3-methyl p-chlorophenol; 2-ethyl-3,5-dimethyl p-chlorophenol; 6-sec-butyl-3-methyl p-chlorophenol; 2-iso-propyl-3,5-dimethyl p-chlorophenol; 6-diethylmethyl-3-methyl p-chlorophenol; 6-iso-propyl-2-ethyl-3-methyl p-chlorophenol; 2-sec-amyl-3,5-dimethyl p-chlorophenol; 2-diethylmethyl-3,5-dimethyl p-chlorophenol; 6-sec-octyl-3-methyl p-chlorophenol; p-chloro-m-cresol p-bromophenol; methyl p-bromophenol; ethyl p-bromophenol; n-propyl p-bromophenol; n-butyl p-bromophenol; n-amyl p-bromophenol; sec-amyl p-bromophenol; n-hexyl p-bromophenol; cyclohexyl p-bromophenol; o-bromophenol; tert-amyl o-bromophenol; n-hexyl o-bromophenol; n-propyl-m,m-dimethyl o-bromophenol; 2-phenyl phenol; 4-chloro-2-methyl phenol; 4-chloro-3-methyl phenol; 4-chloro-3,5-dimethyl phenol; 2,4-dichloro-3,5-dimethylphenol; 3,4,5,6-tetabromo-2-methylphenol-; 5-methyl-2-pentylphenol; 4-isopropyl-3-methylphenol; para-chloro-metaxylenol (PCMX); chlorothymol; phenoxyethanol; phenoxyisopropanol; and 5-chloro-2-hydroxydiphenylmethane.

Resorcinol and its derivatives can also be used as antimicrobial agents. Examples of resorcinol derivatives include, but are not limited to: methyl resorcinol; ethyl resorcinol; n-propyl resorcinol; n-butyl resorcinol; n-amyl resorcinol; n-hexyl resorcinol; n-heptyl resorcinol; n-octyl resorcinol; n-nonyl resorcinol; phenyl resorcinol; benzyl resorcinol; phenylethyl resorcinol; phenylpropyl resorcinol; p-chlorobenzyl resorcinol; 5-chloro-2,4-dihydroxydiphenyl methane; 4′-chloro-2,4-dihydroxydiphenyl methane; 5-bromo-2,4-dihydroxydiphenyl methane; and 4′-bromo-2,4-dihydroxydiphenyl methane.

Examples of bisphenolic antimicrobial agents that can be used in the disclosure include, but are not limited to: 2,2′-methylene bis-(4-chlorophenol); 2,4,4′trichloro-2′-hydroxy-diphenyl ether, which is sold by Ciba Geigy, Florham Park, N.J. under the tradename Triclosan®; 2,2′-methylene bis-(3,4,6-trichlorophenol); 2,2′-methylene bis-(4-chloro-6-bromophenol); bis-(2-hydroxy-3,5-dichlorop-henyl) sulphide; and bis-(2-hydroxy-5-chlorobenzyl)sulphide.

Examples of benzoic esters (parabens) that can be used in the disclosure include, but are not limited to: methylparaben; propylparaben; butylparaben; ethylparaben; isopropylparaben; isobutylparaben; benzylparaben; sodium methylparaben; and sodium propylparaben.

Examples of halogenated carbanilides that can be used in the disclosure include, but are not limited to: 3,4,4′-trichlorocarbanilides, such as 3-(4-chlorophenyl)-1-(3,4-dichlorphenyl)urea sold under the tradename Triclocarban® by Ciba-Geigy, Florham Park, N.J.; 3-trifluoromethyl-4,4′-dichlorocarbanilide; and 3,3′,4-trichlorocarbanilide.

Examples of polymeric antimicrobial agents that can be used in the disclosure include, but are not limited to: polyhexamethylene biguanide hydrochloride; and poly(iminoimidocarbonyl iminoimidocarbonyl iminohexamethylene hydrochloride), which is sold under the tradename Vantocil® IB.

Examples of thiazolines that can be used in the disclosure include, but are not limited to that sold under the tradename Micro-Check®; and 2-n-octyl-4-isothiazolin-3-one, which is sold under the tradename Vinyzene® IT-3000 DIDP.

Examples of trichloromethylthioimides that can be used in the disclosure include, but are not limited to: N-(trichloromethylthio)phthalimide, which is sold under the tradename Fungitrol®; and N-trichloromethylthio-4-cyclohexene-1,2-dicarboximide, which is sold under the tradename Vancide®.

Examples of natural antimicrobial agents that can be used in the disclosure include, but are not limited to, oils of: anise; lemon; orange; rosemary; wintergreen; thyme; lavender, cloves; hops; tea tree; citronella; wheat; barley; lemongrass; cedar leaf; cedarwood; cinnamon; fleagrass; geranium; sandalwood; violet; cranberry; eucalyptus; vervain; peppermint; gum benzoin; basil; fennel; fir, balsam; menthol; ocmea origanuin; hydastis; carradensis; Berberidaceac daceae; Ratanhiae longa; and Curcuma longa. Also included in this class of natural antimicrobial agents are the key chemical components of the plant oils which have been found to provide antimicrobial benefit. These chemicals include, but are not limited to: anethol; catechole; camphene; thymol; eugenol; eucalyptol; ferulic acid; farnesol; hinokitiol; tropolone; limonene; menthol; methyl salicylate; carvacol; terpineol; verbenone; berberine; ratanhiae extract; caryophellene oxide; citronellic acid; curcumin; nerolidol; and geraniol.

Examples of metal salts that can be used in the disclosure include, but are not limited to, salts of metals in groups 3a-5a, 3b-7b, and 8 of the periodic table. Examples of metal salts include, but are not limited to, salts of: aluminum; zirconium; zinc; silver; gold; copper, lanthanum; tin; mercury; bismuth; selenium; strontium; scandium; yttrium; cerium; praseodymiun; neodymium; promethum; samarium; europium; gadolinium; terbium; dysprosium; holmium; erbium; thalium; ytterbium; lutetium; and mixtures thereof. An example of the metal-ion based antimicrobial agent is sold under the tradename HealthShield®, and is manufactured by HealthShield Technology, Wakefield, Mass.

Examples of broad-spectrum antimicrobial agents that can be used in the disclosure include, but are not limited to, those that are recited in other categories of antimicrobial agents herein.

Additional antimicrobial agents that can be used in the methods of the disclosure include, but are not limited to: pyrithiones, and in particular pyrithione-including zinc complexes such as that sold under the tradename Octopirox®; dimethyidimethylol hydantoin, which is sold under the tradename Glydant®; methylchloroisothiazolinone/methylisothiazolinone, which is sold under the tradename Kathon CG®; sodium sulfite; sodium bisulfite; imidazolidinyl urea, which is sold under the tradename Germall 115®; diazolidinyl urea, which is sold under the tradename Germall 11®; benzyl alcohol v2-bromo-2-nitropropane-1,3-diol, which is sold under the tradename Bronopol®; formalin or formaldehyde; iodopropenyl butylcarbamate, which is sold under the tradename Polyphase P100®; chloroacetamide; methanamine; methyldibromonitrile glutaronitrile (1,2-dibromo-2,4-dicyanobutane), which is sold under the tradename Tektamer®; glutaraldehyde; 5-bromo-5-nitro-1,3-dioxane, which is sold under the tradename Bronidox®; phenethyl alcohol; o-phenylphenol/sodium o-phenylphenol sodium hydroxymethylglycinate, which is sold under the tradename Suttocide A®; polymethoxy bicyclic oxazolidine; which is sold under the tradename Nuosept C®; dimethoxane; thimersal; dichlorobenzyl alcohol; captan; chlorphenenesin; dichlorophene; chlorbutanol; glyceryl laurate; halogenated diphenyl ethers; 2,4,4′-trichloro-2′-hydroxy-diphenyl ether, which is sold under the tradename Triclosan® and is available from Ciba-Geigy, Florham Park, N.J.; and 2,2′-dihydroxy-5,5′-dibromo-diphenyl ether.

### (4) Optical Properties of the Biophotonic Compositions

In certain embodiments, biophotonic compositions of the present disclosure are substantially transparent or translucent. The % transmittance of the biophotonic composition can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the biophotonic composition is measured with the chromophore omitted. As transmittance is dependent upon thickness, the thickness of each sample can be measured with calipers prior to loading in the spectrophotometer. Transmittance values can be normalized according to

$${F}_{T - corr} ( λ , {t}_{2} ) = {[ {e}^{- {σ}_{t}} ( λ ) {t}_{1} ]}^{\frac{{t}_{2}}{{t}_{1}}} = {[ {F}_{T - corr} ( λ , {t}_{1} ) ]}^{\frac{{t}_{2}}{{t}_{1}}} ,$$

where t₁=actual specimen thickness, t₂=thickness to which transmittance measurements can be normalized. In the art, transmittance measurements are usually normalized to 1 cm.

In certain embodiments, the biophotonic compositions are substantially opaque. In these embodiments, the biophotonic compositions may include light transmitting structures such as fibres, particles, networks, which are made of materials which can transmit light. The light transmitting structures can be waveguides such as optical fibres.

In some embodiments, the biophotonic composition has a transmittance that is more than about 20%, 25%, 30%, 35%, 40%, 45%, 50%, 55%, 60%, 65%, 70%, or 75% within the visible range. In some embodiments, the transmittance exceeds 40%, 41%, 42%, 43%, 44%, or 45% within the visible range.

### (5) Forms of the Biophotonic Compositions

The biophotonic compositions of the present disclosure may be a liquid, a gel, a cream, a paste, a putty, a semi-solid, or a solid. Biophotonic compositions in the liquid, gel, cream, paste or putty form can be applied by spreading, spraying, smearing, dabbing or rolling the composition on the target tissue. Biophotonic compositions of the putty, semi-solid or solid forms may be deformable. They may be elastic or non-elastic (i.e. flexible or rigid). The biophotonic compositions, for example, may be in a peel-off form (‘peelable’) to provide ease and speed of use. In certain embodiments, the tear strength and/or tensile strength of the peel-off form is greater than its adhesion strength. This may help handleability of the material. It will be recognized by one of skill in the art that the properties of the peel-off biophotonic composition such as cohesiveness, flexibility, elasticity, tensile strength, and tearing strength, can be determined and/or adjusted by methods known in the art such as by selecting suitable thickening agents and adapting their relative ratios.

The biophotonic composition comprising halogen may be in a pre-formed shape. In certain embodiments, the pre-formed shape is in the form of, including, but not limited to, a film, a face mask, a patch, a dressing, or bandage. The biophotonic composition can be configured with a shape and/or size for application to a desired portion of a subject's body. For example, the biophotonic composition can be shaped and sized to correspond with a desired portion of the body to receive the biophotonic treatment. Such a desired portion of the body can be selected from, but not limited to, the group consisting of a skin, head, forehead, scalp, nose, cheeks, lips, ears, face, neck, shoulder, arm pit, arm, elbow, hand, finger, abdomen, chest, stomach, back, buttocks, sacrum, genitals, legs, knee, feet, toes, nails, hair, any boney prominences, and combinations thereof, and the like. Thus, the biophotonic composition of the disclosure can be shaped and sized to be applied to any portion of tissue on a subject's body. For example, the biophotonic composition can be provided in the form of sock, hat, glove or mitten.

In certain aspects, the biophotonic composition forms part of a composite and can include fibres, particulates, non-biophotonic layers or biophotonic layers with the same or different compositions.

The biophotonic compositions of the present disclosure may have a thickness of, or be applied with a thickness of from about 0.1 mm to about 50 mm, about 0.5 mm to about 20 mm, or about 1 mm to about 10 mm. It will be appreciated that the thickness of the biophotonic compositions will vary based on the intended use. In some embodiments, the biophotonic composition has a thickness of from about 0.1-1 mm. In some embodiments, the biophotonic composition has a thickness of about 0.5-1.5 mm, about 1-2 mm, about 1.5-2.5 mm, about 2-3 mm, about 2.5-3.5 mm, about 3-4 mm, about 3.5-4.5 mm, about 4-5 mm, about 4.5-5.5 mm, about 5-6 mm, about 5.5-6.5 mm, about 6-7 mm, about 6.5-7.5 mm, about 7-8 mm, about 7.5-8.5 mm, about 8-9 mm, about 8.5-9.5, about 9-10 mm, about 10-11 mm, about 11-12 mm, about 12-13 mm, about 13-14 mm, about 14-15 mm, about 15-16 mm, about 16-17 mm, about 17-18 mm, about 18-19 mm, about 19-20 mm, about 20-22 mm, about 22-24 mm, about 24-26 mm, about 26-28 mm, about 28-30 mm, about 30-35 mm, about 35-40 mm, about 40-45 mm, about 45-50 mm.

### (6) Methods of Use

The biophotonic composition comprising halogen of the present disclosure may have cosmetic and/or medical benefits. They can be used to promote skin rejuvenation and skin conditioning, promote the treatment of a skin disorder such as acne, eczema or psoriasis, promote tissue repair, and promote wound healing including periodontitis pockets. They can be used to treat acute inflammation. Acute inflammation can present itself as pain, heat, redness, swelling and loss of function, and includes inflammatory responses such as those seen in allergic reactions such as those to insect bites e.g.; mosquito, bees, wasps, poison ivy, or post-ablative treatment.

The biophotonic composition of the present disclosure may have cosmetic and/or medical benefits in the veterinary field, pertaining to the care of animals, such as, but not limited to, cats, dogs, horses, sheep, goat, cows, pigs, hamsters, guinea pig, rabbits. They can be used to promote treatment of the skin of an animal and/or treat animal such as, but not limited to, constant scratching, licking and chewing at the skin, Scabs, Redness or inflammation, round, scaly patches on the face and paws, dryness, flaky, irritated skin, rashes, swellings, lumps or skin discoloration, drainage of blood or pus. They can be used to treat acute inflammation in animals. Acute inflammation in animals can present itself as pain, heat, redness, swelling and loss of function, and includes inflammatory responses such as those seen in allergic reactions such as those to insect bites e.g.; mosquito, bees, wasps, poison ivy, or post-ablative treatment.

Accordingly, in certain embodiments, the present disclosure provides a method for treating acute inflammation, the method comprising: applying a biophotonic composition comprising halogen of the present disclosure to the area of the skin or tissue in need of treatment, and illuminating the biophotonic composition with light having a wavelength that overlaps with an absorption spectrum of the chromophore(s) present in the biophotonic composition.

In certain embodiments, the present disclosure provides a method for providing skin rejuvenation or for improving a skin condition, treating a skin disorder, preventing or treating scarring, debriding wounds and/or skin, and/or accelerating wound healing and/or tissue repair, the method comprising: applying a biophotonic composition comprising halogen of the present disclosure to the area of the skin or tissue in need of treatment, and illuminating the biophotonic composition with light having a wavelength that overlaps with an absorption spectrum of the chromophore(s) present in the biophotonic composition.

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more photoactivators present in the composition. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 to 800 nm. In another embodiment, the source of the actinic light is a source of visible light having a wavelength between about 400 and 600 nm. In another embodiment, the source of the actinic light is a source of visible light having a wavelength between about 400 and 700 nm or about 400 nm to about 750 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm², or about 30 to about 150 mW/cm². Suitable power density for laser light sources are in the range from about 0.5 mW/cm² to about 0.8 mW/cm².

In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic composition. In certain embodiments, the light at the subject's skin is between about 1-40 mW/cm², or 20-60 mW/cm², or 40-80 mW/cm², or 60-100 mW/cm², or 80-120 mW/cm², or 100-140 mW/cm², or 30-180 mW/cm², or 120-160 mW/cm², or 140-180 mW/cm², or 160-200 mW/cm², or 110-240 mW/cm², or 110-150 mW/cm², or 190-240 mW/cm².

The activation of the chromophore(s) within the biophotonic composition may take place almost immediately on illumination (femto- or pico seconds). A prolonged exposure period may be beneficial to exploit the synergistic effects of the absorbed, reflected and reemitted light of the biophotonic composition of the present disclosure and its interaction with the tissue being treated. In one embodiment, the time of exposure to actinic light of the tissue or skin or biophotonic composition is a period between 1 minute and 5 minutes. In another embodiment, the time of exposure to actinic light of the tissue or skin or biophotonic composition is a period between 1 minute and 5 minutes. In some other embodiments, the biophotonic composition is illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 5-9 minutes, about 5-8 minutes, about 10-15 minutes, about 15-20 minutes, about 20-25 minutes, or about 20-30 minutes. The treatment time may range up to about 90 minutes, about 80 minutes, about 70 minutes, about 60 minutes, about 50 minutes, about 40 minutes, about 30 minutes or about 20 minutes. It will be appreciated that the treatment time can be adjusted in order to maintain a dosage by adjusting the rate of fluence delivered to a treatment area. For example, the delivered fluence may be about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm². The delivery fluence may also be adjusted in terms of levels of singlet oxygen released.

In certain embodiments, the biophotonic composition may be re-illuminated at certain intervals, such as 1, 2, 3, 4, 5, 6, 7, 8, 9, 10, 11, 12, 24, 36, or 48 hours. In yet another embodiment, the source of actinic light is in continuous motion over the treated area for the appropriate time of exposure. In yet another embodiment, the biophotonic composition may be illuminated until the biophotonic composition is at least partially photobleached or fully photobleached.

In certain embodiments, the chromophore(s) in the composition can be photoexcited by ambient light including from the sun and overhead lighting. In certain embodiments, the chromophore(s) can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods of the present disclosure, the biophotonic composition may be removed from the skin following application of light. In some embodiments the biophotonic composition is peeled off, or is washed off, the tissue being treated after a treatment time. In other embodiments, the biophotonic composition is left in place on the tissue for an extended period of time and re-activated with direct or ambient light at appropriate times to treat the condition.

(a) Dermatological and Tissue-Related Uses

In certain embodiments of the method of the present disclosure, the biophotonic composition can be applied to the tissue, such as on the face or wound, once, twice, three times, four times, five times or six times a week, daily, or at any other frequency. The total treatment time can be one week, two weeks, three weeks, four weeks, five weeks, six weeks, seven weeks, eight weeks, nine weeks, ten weeks, eleven weeks, twelve weeks, or any other length of time deemed appropriate. In certain embodiments, the total tissue area to be treated may be split into separate areas (cheeks, forehead), and each area treated separately. For example, the composition may be applied topically to a first portion, and that portion illuminated with light, and the biophotonic composition then removed. Then the composition is applied to a second portion, illuminated and removed. Finally, the composition is applied to a third portion, illuminated and removed.

In certain embodiments, the biophotonic composition can be used following wound closure to optimize scar revision. In this case, the biophotonic composition may be applied at regular intervals such as once a week, or at an interval deemed appropriate by the physician or any other health care provider.

In certain embodiments, the biophotonic composition can be used following acne treatment to maintain the condition of the treated skin. In this case, the biophotonic composition may be applied at regular intervals such as once a week, or at an interval deemed appropriate by the physician or any other health care provider.

In certain embodiments, the biophotonic composition comprising halogen can be used following ablative skin rejuvenation treatment to maintain the condition of the treated skin. In this case, the biophotonic composition may be applied at regular intervals such as once a week, or at an interval deemed appropriate by the physician or any other health care provider.

In certain embodiments, the biophotonic composition comprising halogen can be used to debride a wound or to loosen or remove scaley, dry or dead skin. In this case, the biophotonic composition may be applied at regular intervals such as once a week, or at an interval deemed appropriate by the physician or any other health care provider.

In certain embodiments, the biophotonic composition comprising halogen can be used to treat bacterial, viral or fungal infections. In this case, the biophotonic composition may be applied at regular intervals such as once a week, or at an interval deemed appropriate by the physician or any other health care provider.

In the methods of the present disclosure, additional components may optionally be included in the biophotonic compositions or used in combination with the biophotonic compositions. Such additional components include, but are not limited to, healing factors, antimicrobials, oxygen-rich agents, wrinkle fillers such as botox, hyaluronic acid and polylactic acid, fungal, anti-bacterial, anti-viral agents and/or agents that promote collagen synthesis. These additional components may be applied to the skin in a topical fashion, prior to, at the same time of, and/or after topical application of the biophotonic compositions of the present disclosure. Suitable healing factors comprise compounds that promote or enhance the healing or regenerative process of the tissues on the application site. During the photoactivation of a biophotonic composition of the present disclosure, there may be an increase of the absorption of molecules of such additional components at the treatment site by the skin or the mucosa. In certain embodiments, an augmentation in the blood flow at the site of treatment can observed for a period of time. An increase in the lymphatic drainage and a possible change in the osmotic equilibrium due to the dynamic interaction of the free radical cascades can be enhanced or even fortified with the inclusion of healing factors. Healing factors may also modulate the biophotonic output from the biophotonic composition such as photobleaching time and profile, or modulate leaching of certain ingredients within the composition. Suitable healing factors include, but are not limited to glucosamines, allantoins, saffron, agents that promote collagen synthesis, anti-fungal, anti-bacterial, anti-viral agents and wound healing factors such as growth factors.

(i) Skin Rejuvenation

The biophotonic composition comprising halogen of the present disclosure may be useful in promoting skin rejuvenation or improving skin condition and appearance. The dermis is the second layer of skin, containing the structural elements of the skin, the connective tissue. There are various types of connective tissue with different functions. Elastin fibers give the skin its elasticity, and collagen gives the skin its strength.

The junction between the dermis and the epidermis is an important structure. The dermal-epidermal junction interlocks forming finger-like epidermal ridges. The cells of the epidermis receive their nutrients from the blood vessels in the dermis. The epidermal ridges increase the surface area of the epidermis that is exposed to these blood vessels and the needed nutrients.

The aging of skin comes with significant physiological changes to the skin. The generation of new skin cells slows down, and the epidermal ridges of the dermal-epidermal junction flatten out. While the number of elastin fibers increases, their structure and coherence decreases. Also the amount of collagen and the thickness of the dermis decrease with the ageing of the skin.

Collagen is a major component of the skin's extracellular matrix, providing a structural framework. During the aging process, the decrease of collagen synthesis and insolubilization of collagen fibers contribute to a thinning of the dermis and loss of the skin's biomechanical properties.

The physiological changes to the skin result in noticeable aging symptoms often referred to as chronological-, intrinsic- and photo-ageing. The skin becomes drier, roughness and scaling increase, the appearance becomes duller, and most obviously fine lines and wrinkles appear. Other symptoms or signs of skin aging include, but are not limited to, thinning and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), an inability to sweat sufficiently in order to cool the skin, unwanted facial hair, freckles, age spots, spider veins, rough and leathery skin, fine wrinkles that disappear when stretched, loose skin, a blotchy complexion.

The dermal-epidermal junction is a basement membrane that separates the keratinocytes in the epidermis from the extracellular matrix, which lies below in the dermis. This membrane consists of two layers: the basal lamina in contact with the keratinocytes, and the underlying reticular lamina in contact with the extracellular matrix. The basal lamina is rich in collagen type IV and laminin, molecules that play a role in providing a structural network and bioadhesive properties for cell attachment.

Laminin is a glycoprotein that only exists in basement membranes. It is composed of three polypeptide chains (alpha, beta and gamma) arranged in the shape of an asymmetric cross and held together by disulfide bonds. The three chains exist as different subtypes which result in twelve different isoforms for laminin, including Laminin-1 and Laminin-5.

The dermis is anchored to hemidesmosomes, specific junction points located on the keratinocytes, which consist of α-integrins and other proteins, at the basal membrane keratinocytes by type VII collagen fibrils. Laminins, and particularly Laminin-5, constitute the real anchor point between hemidesmosomal transmembrane proteins in basal keratinocytes and type VII collagen.

Laminin-5 synthesis and type VII collagen expression have been proven to decrease in aged skin. This causes a loss of contact between dermis and epidermis, and results in the skin losing elasticity and becoming saggy.

Recently another type of wrinkles, generally referred to as expression wrinkles, got general recognition. These wrinkles require loss of resilience, particularly in the dermis, because of which the skin is no longer able to resume its original state when facial muscles which produce facial expressions exert stress on the skin, resulting in expression wrinkles.

The biophotonic composition comprising halogen of the present disclosure and methods of the present disclosure promote skin rejuvenation. In certain embodiments, the biophotonic composition and methods of the present disclosure promote skin condition such as skin luminosity, reduction of pore size, reducing blotchiness, making even skin tone, reducing dryness, and tightening of the skin. In certain embodiments, the biophotonic composition and methods of the present disclosure promote collagen synthesis. In certain other embodiments, the biophotonic composition and methods of the present disclosure may reduce, diminish, retard or even reverse one or more signs of skin aging including, but not limited to, appearance of fine lines or wrinkles, thin and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), inability to sweat sufficiently to cool the skin, unwanted facial hair, freckles, age spots, spider veins, rough and leathery skin, fine wrinkles that disappear when stretched, loose skin, or a blotchy complexion. In certain embodiments, the biophotonic composition comprising halogen and methods of the present disclosure may induce a reduction in pore size, enhance sculpturing of skin subsections, and/or enhance skin translucence.

In certain embodiments, the biophotonic composition comprising halogen may be used in conjunction with collagen promoting agents. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication 2009/0069217. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola and lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsin, trypsin, or chymotrypsin; and a mixture containing two or more of those collagens at an arbitrary rate. Collagens extracted from plant sources may also be used.

(ii) Skin disorders

The biophotonic compositions comprising halogen and methods of the present disclosure may be used to treat skin disorders that include, but are not limited to, erythema, telangiectasia, actinic telangiectasia, basal cell carcinoma, contact dermatitis, dermatofibrosarcoma protuberans, genital warts, hidradenitis suppurativa, melanoma, merkel cell carcinoma, nummular dermatitis, molloscum contagiosum, psoriasis, psoriatic arthritis, rosacea, scabies, scalp psoriasis, sebaceous carcinoma, squamous cell carcinoma, seborrheic dermatitis, seborrheic keratosis, shingles, tinea versicolor, warts, skin cancer, pemphigus, sunburn, dermatitis, eczema, rashes, impetigo, lichen simplex chronicus, rhinophyma, perioral dermatitis, pseudofolliculitis barbae, drug eruptions, erythema multiforme, erythema nodosum, granuloma annulare, actinic keratosis, purpura, alopecia areata, aphthous stomatitis, dry skin, chapping, xerosis, ichthyosis vulgaris, fungal infections, herpes simplex, intertrigo, keloids, keratoses, milia, moluscum contagiosum, pityriasis rosea, pruritus, urticaria, and vascular tumors and malformations. Dermatitis includes contact dermatitis, atopic dermatitis, seborrheic dermatitis, nummular dermatitis, generalized exfoliative dermatitis, and statis dermatitis. Skin cancers include melanoma, basal cell carcinoma, and squamous cell carcinoma.

(iii) Acne and Acne Scars

The biophotonic compositions and methods of the present disclosure may be used to treat acne. As used herein, “acne” means a disorder of the skin caused by inflammation of skin glands or hair follicles. The biophotonic compositions and methods of the disclosure can be used to treat acne at early pre-emergent stages or later stages where lesions from acne are visible. Mild, moderate and severe acne can be treated with embodiments of the biophotonic compositions and methods. Early pre-emergent stages of acne usually begin with an excessive secretion of sebum or dermal oil from the sebaceous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone. In the normal sequence of developing acne, hyperkeratinazation of the follicular opening is stimulated, thus completing blocking of the duct. The usual results are papules, pustules, or cysts, often contaminated with bacteria, which cause secondary infections. Acne is characterized particularly by the presence of comedones, inflammatory papules, or cysts. The appearance of acne may range from slight skin irritation to pitting and even the development of disfiguring scars. Accordingly, the biophotonic compositions and methods of the present disclosure can be used to treat one or more of skin irritation, pitting, development of scars, comedones, inflammatory papules, cysts, hyperkeratinazation, and thickening and hardening of sebum associated with acne.

Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstrual acne, acne pustulosa, acne scorbutica, acne scrofulosorum, acne urticata, acne varioliformis, acne venenata, propionic acne, acne excoriee, gram negative acne, steroid acne, and nodulocystic acne.

Some skin disorders present various symptoms including redness, flushing, burning, scaling, pimples, papules, pustules, comedones, macules, nodules, vesicles, blisters, telangiectasia, spider veins, sores, surface irritations or pain, itching, inflammation, red, purple, or blue patches or discolorations, moles, and/or tumors.

The biophotonic compositions comprising halogen and methods of the present disclosure may be used to treat various types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

In certain embodiments, the biophotonic composition of the present disclosure is used in conjunction with systemic or topical antibiotic treatment. For example, antibiotics used to treat acne include tetracycline, erythromycin, minocycline, doxycycline, which may also be used with the compositions and methods of the present disclosure. The use of the biophotonic composition can reduce the time needed for the antibiotic treatment or reduce the dosage.

(iv) Tissue Repair, Wound Healing

The biophotonic compositions comprising halogen and methods of the present disclosure may be used to treat wounds, promote wound healing, promote tissue repair and/or prevent or reduce cosmesis including improvement of motor function (e.g. movement of joints). Wounds that may be treated by the biophotonic compositions and methods of the present disclosure include, for example, injuries to the skin and subcutaneous tissue initiated in different ways (e.g., pressure ulcers from extended bed rest, wounds induced by trauma or surgery, burns, ulcers linked to diabetes or venous insufficiency, wounds induced by conditions such as periodontitis) and with varying characteristics. In certain embodiments, the present disclosure provides biophotonic compositions and methods for treating and/or promoting the healing of, for example, fistulas, burns, incisions, excisions, lesions, lacerations, abrasions, puncture or penetrating wounds, surgical wounds, contusions, hematomas, crushing injuries, amputations, sores and ulcers.

Biophotonic compositions and methods of the present disclosure may be used to treat and/or promote the healing of a fistula. A fistula is an abnormal connection between an organ, vessel, or intestine and another structure, and while a fistula is usually caused by injury or surgery, it may also result from an infection or inflammation, and examples of fistulas that may be treated with the biophotonic composition of the present invention include, but are not limited to, a preauricular sinus or cyst, anal fistulas, rectal fistulas, fistulas of the joints, fistulas of the urogenital tract or in relation to the reproductive organs, and fistulas that may occur at any other location on the body.

Biophotonic compositions and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

For example, the present disclosure provides biophotonic compositions and methods for treating and/or promoting healing of a diabetic ulcer. Diabetic patients are prone to foot and other ulcerations due to both neurologic and vascular complications. Peripheral neuropathy can cause altered or complete loss of sensation in the foot and/or leg. Diabetic patients with advanced neuropathy lose all ability for sharp-dull discrimination.

Any cuts or trauma to the foot may go completely unnoticed for days or weeks in a patient with neuropathy. A patient with advanced neuropathy loses the ability to sense a sustained pressure insult, as a result, tissue ischemia and necrosis may occur leading to for example, plantar ulcerations. Microvascular disease is one of the significant complications for diabetics which may also lead to ulcerations. In certain embodiments, biophotonic compositions and methods of treating a chronic wound are provided here in, where the chronic wound is characterized by diabetic foot ulcers and/or ulcerations due to neurologic and/or vascular complications of diabetes.

In other examples, the present disclosure provides biophotonic compositions and methods for treating and/or promoting healing of a pressure ulcer. Pressure ulcers include bed sores, decubitus ulcers and ischial tuberosity ulcers and can cause considerable pain and discomfort to a patient. A pressure ulcer can occur as a result of a prolonged pressure applied to the skin. Thus, pressure can be exerted on the skin of a patient due to the weight or mass of an individual. A pressure ulcer can develop when blood supply to an area of the skin is obstructed or cut off for more than two or three hours. The affected skin area can turn red, become painful and necrotic. If untreated, the skin can break open and become infected. A pressure ulcer is therefore a skin ulcer that occurs in an area of the skin that is under pressure from e.g. lying in bed, sitting in a wheelchair, and/or wearing a cast for a prolonged period of time. Pressure ulcers can occur when a person is bedridden, unconscious, unable to sense pain, or immobile. Pressure ulcers often occur in boney prominences of the body such as the buttocks area (on the sacrum or iliac crest), or on the heels of foot.

In certain other embodiments, the present disclosure provides biophotonic compositions and methods for treating and/or promoting healing, Grade I-IV ulcers. In certain embodiments, the application provides compositions suitable for use with Grade II and Grade III ulcers in particular. Ulcers may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

Wound healing in adult tissues is a complicated reparative process. For example, the healing process for skin involves the recruitment of a variety of specialized cells to the site of the wound, extracellular matrix and basement membrane deposition, angiogenesis, selective protease activity and re-epithelialization.

There are four overlapping phases in the normal wound healing process. First, in the hemostasis and inflammatory phases, which typically occur from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts.

In the proliferative phase, which typically occurs from two days to three weeks, granulation tissue forms, and epithelialization and contraction begin. Fibroblasts, which are key cell types in this phase, proliferate and synthesize collagen to fill the wound and provide a strong matrix on which epithelial cells grow. As fibroblasts produce collagen, vascularization extends from nearby vessels, resulting in granulation tissue. Granulation tissue typically grows from the base of the wound. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility, and are common in granulating wounds.

In the remodeling phase, the final phase of wound healing which can take place from three weeks up to several years, collagen in the scar undergoes repeated degradation and re-synthesis. During this phase, the tensile strength of the newly formed skin increases. However, as the rate of wound healing increases, there is often an associated increase in scar formation. Scarring is a consequence of the healing process in most adult animal and human tissues. Scar tissue is not identical to the tissue which it replaces, as it is usually of inferior functional quality. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion.

In contrast, normal skin consists of collagen fibers arranged in a basket-weave pattern, which contributes to both the strength and elasticity of the dermis. Thus, to achieve a smoother wound healing process, an approach is needed that not only stimulates collagen production, but also does so in a way that reduces scar formation.

The biophotonic compositions and methods of the present disclosure promote the wound healing by promoting the formation of substantially uniform epithelialization; promoting collagen synthesis; promoting controlled contraction; and/or by reducing the formation of scar tissue. In certain embodiments, the biophotonic compositions and methods of the present disclosure may promote wound healing by promoting the formation of substantially uniform epithelialization. In some embodiments, the biophotonic compositions and methods of the present disclosure promote collagen synthesis. In some other embodiments, the biophotonic compositions and methods of the present disclosure promote controlled contraction. In certain embodiments, the biophotonic compositions and methods of the present disclosure promote wound healing, for example, by reducing the formation of scar tissue or by speeding up the wound closure process. In certain embodiments, the biophotonic compositions and methods of the present disclosure promote wound healing, for example, by reducing inflammation. In certain embodiments, the biophotonic composition can be used following wound closure to optimize scar revision. In this case, the biophotonic composition may be applied at regular intervals such as once a week, or at an interval deemed appropriate by the physician.

In the methods of the present disclosure, the biophotonic compositions of the present disclosure may also be used in combination with negative pressure assisted wound closure devices and systems.

In certain embodiments, the biophotonic composition is kept in place for up to one, two or 3 weeks, and illuminated with light which may include ambient light at various intervals. In this case, the composition may be covered up in between exposure to light with an opaque material or left exposed to light. In certain embodiments, the biophotonic composition is removed after each treatment.

(b) Oral Diseases

The biophotonic composition comprising halogen of the present disclosure may be used to treat various oral diseases. Such oral diseases include but are not limited to:

(i) Gingivitis

Gingivitis is a disorder that is defined by the inflammation of the gums, which are characterized by the destruction of the gums, tissue, tooth sockets, and ligaments which create the structure that holds the teeth in place.

The symptoms of gingivitis include swollen gums, mouth sores, a bright red or purple appearance to the gums, shiny gums, gums that are painless except when touched, and bleeding gums. Often the first signs of gingivitis have no symptoms except for visual symptoms and is likely only to be diagnosed by a dental professional.

(ii) Periodontal Disease

Periodontal disease may lead to severe gingivitis and can cause gums to bleed, ooze pus, is highly painful, and often leads to premature tooth loss. While most developed nations have fewer cases of periodontal disease, it is none the less a condition that occurs with a great degree of frequency due to a lack of availability of affordable professional dental care for a significant percentage of a population regardless of a country's economic position and rank.

Periodontal disease is more prevalent in developing nations and in most cases, a professional cleaning and antibiotics may ameliorate most cases of periodontal disease. However, if left untreated the infection can spread throughout the body and can lead to serious health complications.

Symptoms of periodontal disease include painful gums, bad breath (halitosis), a foul taste to the mouth, fever, gums that bleed with only mild amounts of pressure, crater sized canker sores between the teeth and gums, swollen lymph nodes around the head, neck, or jaw, a gray film on the gums, red gums, swollen gums, and pain when eating and swallowing.

(iii) Periodontitis

Periodontitis or Pyorrhea alveolaris is the inflammation of the periodontium which comprises tissues supporting the teeth in the oral cavity. Parts included in the periodontium are the gingiva (gum tissue), the alveolar bone which are sockets where teeth are attached, the cementum or outer layer of teeth roots and the periodontal ligaments or PDL composed of connective tissue fibers linking the gingival and cementum to the alveolar bone. The condition is described as the progressive loss of bone around teeth leading to loose teeth or loss of teeth if left unattended. There are different causes for the disease in which bacteria is the most common. Periodontitis is considered as an advanced phase of gum disease since it already involves bone loss in the area. It is the effect of mild gingivitis being left untreated. Due to the presence of bacterial infection, the body can also respond negatively to it leading to further complications. The condition is one of the leading causes of tooth loss among adults, affecting around 50% of adults over the age of 30.

Signs and symptoms arise due to the unstable anchoring of teeth as well as the presence of microorganisms. Gums occasionally or frequently bleed or turn red while brushing teeth, using dental floss, biting into food, chewing or touching with fingers. Gums swell or develop pus occasionally as well. The affected individual likely has halitosis or bad breath and may have a lingering metallic or tinny taste inside the mouth. Teeth seem longer and sharper due to gingival recession which partly may also be caused by hard brushing. If enzymes such as collagenases have begun destroying collagen, the person will have deep pockets (termed perio-pockets) between the teeth and gums.

During the early stages of periodontal disease, only a few signs and symptoms may be noticeable. Aggressive periodontitis may affect younger individuals and can occur in episodes. Some episodes may present very mild symptoms while others may be very severe. The signs and symptoms especially in the case of chronic periodontitis are usually progressive in nature.

(iv) Oral Thrush

Oral thrush is the condition where the fungus Candida albicans grows rapidly and uncontrollably in the mouth. The bacterium known as flora keeps the growth of Candida albicans under control in a healthy body. Oral thrush presents with creamy white paste that covers the tongue, and can spread rapidly to the roof of the mouth, gums, back of the throat, tonsils, and the inside of the cheeks. Babies, toddlers, older adults, and patients whose immune systems have been somehow compromised are most likely to be afflicted with oral thrush.

Symptoms of oral thrush begin with a white pasty covering over the tongue and inside of the cheeks. As the oral thrush continues to develop, it can cause a mild amount of bleeding if the tongue is scraped or when the patient brushes their teeth. These symptoms may develop very quickly, and the thrush can last for months. If the lesions of oral thrush spread down the esophagus, the patient may develop addition symptoms such as difficulty swallowing, the sensation of food being caught in the throat or the middle of the chest, and a fever should the infection continue to spread past the esophagus.

(v) Lichen Planus

Lichen planus is most often defined as an oral disease that affects the lining of the mouth with inflammation. Lichen planus is most often recognized as a rash that irritates the tissue of the oral cavity. Most patients come down with their first case between the age of 45 and 60, although the incidence in younger patients has been slowly increasing. While lichen planus is most often associated with the interior of the cheeks, many cases will find the entire mouth is affected, including the gums, the tongue, the lips, and in rare cases, the throat or esophagus. Lichen planus also occurs on the skin, as a skin disease, and often must be referred to specifically as skin lichen planus to differentiate between the oral type.

Lichen planus is a self-contained disease that can last for weeks, months, and in some cases, years. It is not contagious. It is often mistaken for genital diseases, as the genitalia are often the most noticeably affected during the early development stage. Because the symptoms and outbreaks occur rapidly and then disappear, often for weeks, treatment is difficult. While some patients find great relief in cool compresses or tub soaks and cool baths, most patients require medical treatment in order to relieve their symptoms.

(vi) Stomatitis

Stomatitis basically means inflammation of the mouth, but more specifically, stomatitis is the inflammation of the mucous lining of the mouth which may include the gums, tongue, cheeks, lips and the floor or roof of the mouth. There are different types of stomatitis and classification is based on how the disease was acquired by a person. The two types of stomatitis are contact stomatitis and aphthous stomatitis. Contact stomatitis is an inflammation of the oral mucosa caused by coming in contact with allergens or irritants. It is classified by its pattern of distribution, etiologic factors, and clinical features. There some cases of contact stomatitis that are left undetected because of the lack of clinical signs. Anybody can have contact stomatitis regardless of race, age and sex. Although it has been observed that it is more common in the elders.

Aphthous stomatitis, also known as canker sore or aphthous ulcers, has an unknown etiology. Just like contact stomatitis, canker sore affects the oral mucosa. An aphthous ulcer is a type of oral ulcer, which presents as a painful open sore inside the mouth or upper throat (including the uvula) caused by a break in the mucous membrane. The condition is also known as Sutton's Disease, especially in the case of major, multiple, or recurring ulcers. The ulcers can be described as shallow, discrete, and painful and are usually visible on the mucous membranes that are unattached. This type of stomatitis, just like contact stomatitis, is self-limited and do not usually cause complications. The normal size of ulcers may last for 1 to 2 weeks but larger ulcers may last for months.

(vii) Herpes Simplex Lesions

Herpes simplex is a viral disease caused by herpes simplex viruses; both herpes simplex virus 1 (HSV-1) and herpes simplex virus 2 (HSV-2) cause herpes simplex. Infection with the herpes virus is categorized into one of several distinct disorders based on the site of infection. Oral Herpes, the visible symptoms of which are colloquially called cold sores, and infects the face and mouth. Oral herpes is the most common form of herpes simplex virus infection.

(viii) Other Oral Inflammatory Lesions

The present invention may be used to treat other types of oral inflammation, including but not limited to, oral mucositis, oral ulcers caused by viral, bacterial, fungal or protozoan infections, or caused by disorders of the immune system (immunodeficiency, autoimmunity, or allergy). Also included is oral submucous fibrosis, a chronic debilitating disease of the oral cavity characterized by inflammation and progressive fibrosis of the submucosal tissues. Also included is glossitis, an inflammation or infection of the tongue. It causes the tongue to swell and change color.

(c) Bone Regeneration

The biophotonic compositions comprising halogen of the present disclosure may be used for bone reconstruction and/or regeneration. Without being bound by theory, the compositions of the disclosure may help promote the growth, recruitment and survival of bone tissue at a particular site. In use, the composition may be implanted at a site at which bone growth is desired, e.g. to treat a disease, defect or location of trauma, and/or to promote artificial arthrodesis. Bone repair sites that can be treated with the composition of the disclosure include, but are not limited to, those resulting from injury, defects brought about during the course of surgery, infection, malignancy or developmental malformation. The compositions may be used in a wide variety of orthopedic, periodontal, neurosurgical and oral and maxillofacial surgical procedures including, but not limited to: the repair of simple and compound fractures and non-unions; external and internal fixations; joint reconstructions such as arthrodesis; general arthroplasty; cup arthroplasty of the hip; femoral and humeral head replacement; femoral head surface replacement and total joint replacement; repairs of the vertebral column including spinal fusion and internal fixation; tumor surgery, e.g., deficit filing; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay osteoimplants; implant placement and revision; sinus lifts; and cosmetic enhancement. For any of these potential applications, compositions of the disclosure may be applied directly to a site where bone reconstruction is needed. Accessing this site may, in some cases, require surgical intervention to expose the site. However, in some cases, the site is already exposed or can be accessed without the need for surgical intervention.

A bone disease or disorder that may be treated using the composition of the present disclosure include genetic diseases, congenital abnormalities, fractures, iatrogenic defects, bone cancer, bone metastases, inflammatory diseases (e.g. rheumatoid arthritis), autoimmune diseases, metabolic diseases, and degenerative bone disease (e.g., osteoarthritis). In certain embodiments, the compositions are formulated for the repair of a simple fracture, compound fracture, or non-union; as an external fixation device or internal fixation device; for joint reconstruction, arthrodesis, arthroplasty, or cup arthroplasty of the hip; for femoral or humeral head replacement; for femoral head surface replacement or total joint replacement; for repair of the vertebral column, spinal fusion or internal vertebral fixation; for tumor surgery, for deficit filling; for discectomy; for laminectomy; for excision of spinal tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for sinus lift; for a cosmetic procedure; for revision surgery; for revision surgery of a total joint arthroplasty; and for the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones, or metatarsal bones. The composition may be made flowable before it is administered to a subject to allow for the composition to fit into irregularly shaped sites. In certain embodiments, the composition may be injected or extruded into a tissue site (e.g., a bony defect or bone cavity). For example, the composition may be injected using a needle and syringe. The syringe may be driven by hand or mechanically. In some embodiments, the mixture may be injected percutaneously. A bony injection site may be some distance from the skin, necessitating a longer needle. In other embodiments, the injection site may be exposed, for example, during surgery. In these cases a very short cannula may suffice for delivery of the mixture, and a wider bore cannula may be appropriate.

(d) Rare and Orphan Diseases

Rare diseases in dermatology for which the invention may be used to treat or alleviate one or more symptoms thereof may include, but are not limited to, CHILD syndrome (Congenital hemidysplasia with ichthyosiform erythroderma and limb defects) and in particular the ichthyosiform erythroderma aspect of CHILD syndrome; dermatomyositis; hidradenitis suppurativa; acquired ichthyosis as well as hereditary ichthyosis; lichen myxedematosus and scleromyxedema; pemphigus; and porphyria disorders.

Rare diseases involving bone and/or connective tissue maladies for which the invention may be used to treat or alleviate one or more symptoms thereof may include, but are not limited to, Ehlers-Danlos syndrome and other rare diseases manifested by a collagen production and/or deposition abnormality, cutis hyperelastica; eosinophilic fasciitis; osteogenesis imperfecta; scelroderma; and Winchester syndrome.

### (7) Kits

The present disclosure also provides kits containing the biophotonic compositions of the present disclosure and/or kits providing any of the components required for preparing biophotonic compositions of the present disclosure.

In some embodiments, the kit includes a biophotonic composition of the present disclosure. In some embodiments, the kit includes containers comprising the components that can be used to make the biophotonic composition of the present disclosure. The different components making up the biophotonic compositions of the present disclosure may be provided in separate containers. For example, the oxidant, such as peroxide or peroxide precursor of the biophotonic composition may be provided in a container separate from the chromophore. Examples of such containers are dual chamber syringes, dual chamber containers with removable partitions, sachets with pouches, and multiple-compartment blister packs. Another example is one of the components being provided in a syringe which can be injected into a container of another component.

In other embodiments, the kit comprises a systemic drug for augmenting the treatment of the biophotonic composition of the present disclosure. For example, the kit may include a systemic or topical antibiotic, hormone treatment (e.g. for acne treatment or wound healing), or a negative pressure device.

In certain embodiments, the kit comprises a first component comprising at least one chromophore; a second component comprising KI, or KCl, or KBr, or CsBr, or MgBr₂, or ZnBr₂, or NaF, or NaCl, or NaBr, or I₂, or I₃⁻, or Br₂, or Cl₂, or a combination thereof; a third component comprising an oxidant, such as peroxide or a peroxide precursor; and a fourth component comprising a carrier; wherein one or more of the components may come in separate containers within the kit. The kit may also include instructions for use. The carrier may be included together with any of the other components.

In some embodiments, the kit comprises a means for applying the components of the biophotonic compositions such as a spatula, a syringe, or the like.

In certain aspects, there is provided a container comprising a chamber for holding a biophotonic composition, and an outlet in communication with the chamber for discharging the biophotonic composition from the container, wherein the biophotonic composition comprises at least one chromophore. In certain embodiments, the chamber is partitioned such that the chromophore, the peroxide or peroxide precursor and the halogen are kept in separate compartments until discharged from the container or during discharging from the container.

In certain embodiments, the kit comprises a first component comprising the biophotonic composition and the second component comprises a dressing or a mask. The dressing or mask may be a porous or semi-porous structure for receiving the biophotonic composition. The dressing or mask may also comprise woven or non-woven fibrous materials. The biophotonic composition or its precursor can be incorporated, such as by injection, into the dressing.

In certain embodiments of the kit, the kit may further comprise a light source such as a portable light with a wavelength appropriate to activate the chromophore the biophotonic composition. The portable light may be battery operated or re-chargeable. The light source may comprise LEDs.

Written instructions on how to use the biophotonic compositions in accordance with the present disclosure may be included in the kit, or may be included on or associated with the containers comprising the compositions or components making up the biophotonic compositions of the present disclosure. The instructions can include information on how to form the biophotonic composition from the individual components or biophotonic composition precursors provided with the kit.

Identification of equivalent biophotonic compositions, methods and kits are well within the skill of the ordinary practitioner and would require no more than routine experimentation, in light of the teachings of the present disclosure.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application.

Practice of the disclosure will be still more fully understood from the following examples, which are presented herein for illustration only and should not be construed as limiting the disclosure in any way.

### EXAMPLES

### Example 1—Photobleaching in Aqueous Solution

In this experiment, a first aqueous solution comprising 109 μg/g of Eosin Y and 12% urea peroxide (UP) was prepared. A second aqueous solution comprising 109 μg/g of Eosin Y, 12% urea peroxide (UP) and 200 ppm KI was also prepared. The two aqueous solutions were then illuminated with blue light (5 cm distance) for 10 minutes. The fluorescence was measured and recorded by a spectrophotometer.

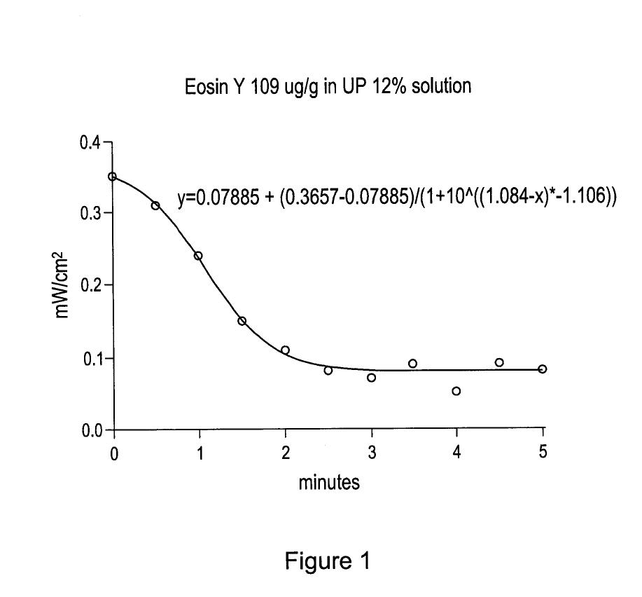

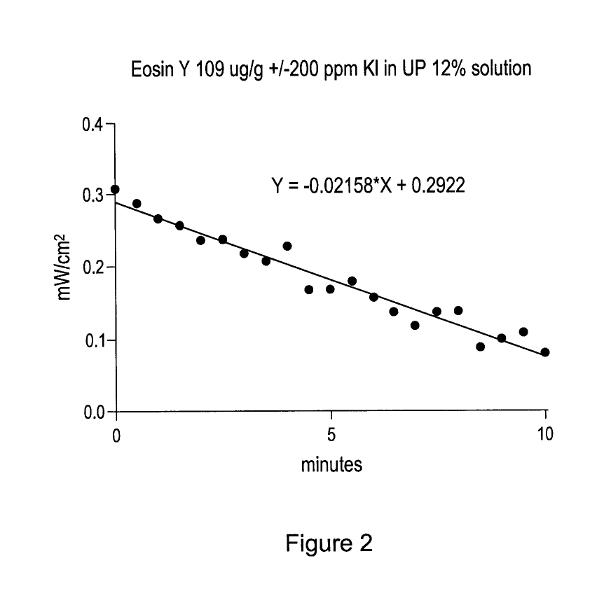

FIGS. 1 and 2 show the peak fluorescence emission of the solutions. From the results, it can be seen that the photobleaching profile over time of the Eosin Y in the KI-containing solution was extended in comparison to the solution that lacked KI; the addition of KI to the chromophore and peroxide mixture prolonged the time taken to photobleach the chromophore.

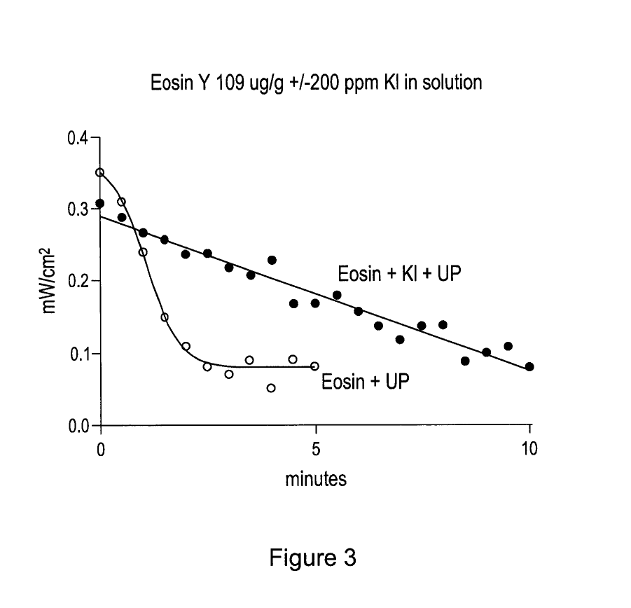

FIG. 3 shows FIGS. 1 and 2 overlaid.

### Example 2—Photobleaching in Carbopol Gel

In this experiment, a first carbomer gel comprising 109 μg/g of Eosin Y and 12% urea peroxide (UP) was prepared. A second carbomer gel comprising 109 μg/g of Eosin Y, 12% urea peroxide (UP) and 200 ppm KI was also prepared. The two gels were then illuminated with blue light (5 cm distance) for 10 minutes. The fluorescence was measured and recorded by a spectrophotometer.

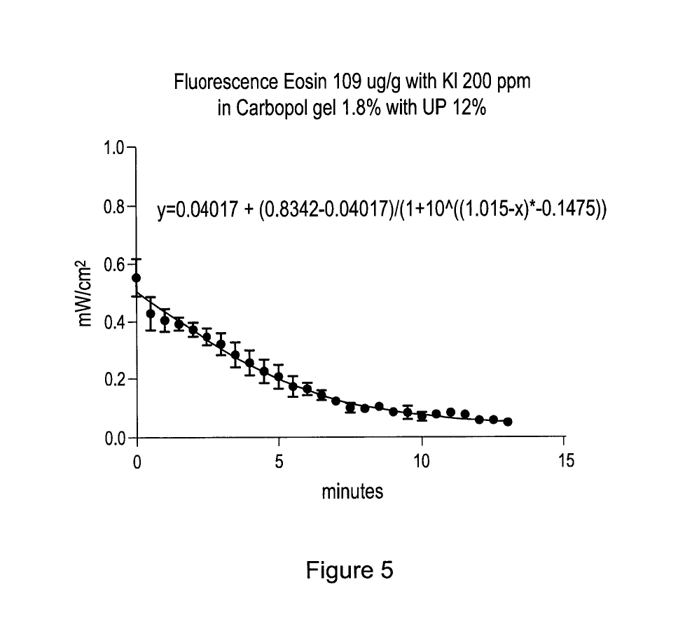

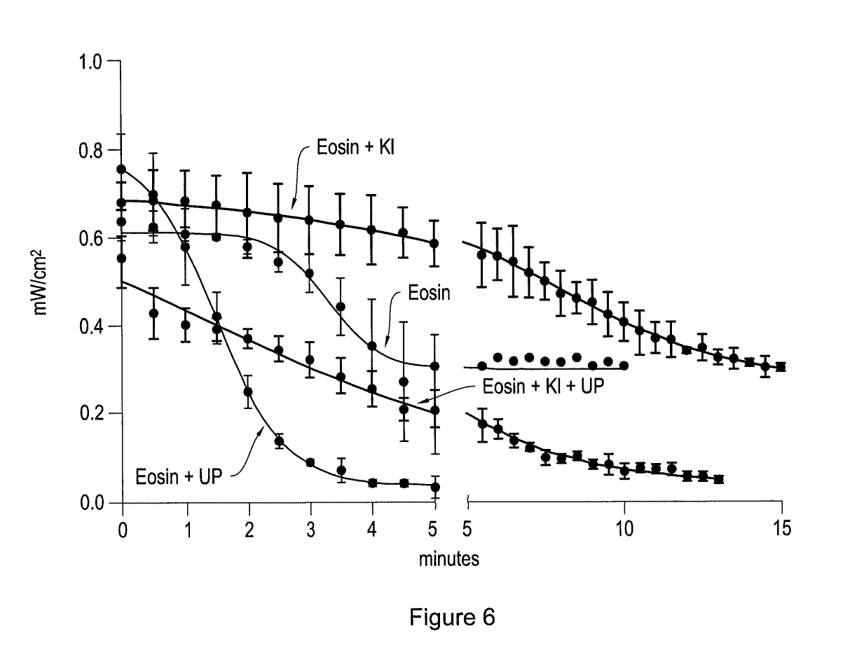

FIGS. 4 and 5 show the peak fluorescence emission of the gels. It can be seen that the KI changes the photobleaching profile over time of the Eosin Y; the addition of KI to the chromophore and peroxide mixture prolonged the time taken to photobleach the chromophore. FIG. 6 is a graph showing the curves of FIGS. 4 and 5 overlaid.

### Example 3—Singlet Oxygen Generation

Single oxygen generation by the compositions of Examples 1 and 2 were evaluated using a pulsed laser method. Each composition sample was excited by a laser (Continuum Surelite SL II-10) configured for the third harmonic at 355 nm and equipped with an Optical Parametric Oscillator (Continuum Surelite OPO Plus) tuned for 450 nm, and a Monochromator (Spectral Products CM-110 1/8m). The emitted fluorescence was collected using a NIR sensitive detector (photomultiplier system Hamamatsu H10330-75). For lifetime measurements, Time-Correlated Single Photon Counting (TCSPC) was used for data acquisition. The short-lived fluorescence data points were plotted, and used to calculate the relative levels and average lifetime of singlet oxygen generated after the excitation pulses. The results are summarized in Table 1 below.

TABLE 1
Singlet oxygen measurements taken immediately after mixing
aqueous solutions containing Eosin Y, with and without peroxide
(urea peroxide or hydrogen peroxide), and with and without
KI, according to embodiments of the present disclosure.

| | Singlet oxygen | Lifetime |
|---|---|---|
| Sample | measurement | (microseconds) |
| Eosin + KI 200 ppm | 0.60; 0.60 | 4.2; 4.0 |
| Eosin + KI 200 ppm + | 1.2; 1.05 | 3.25; 3.3 |
| Urea Peroxide 12% | | |
| Eosin + Urea Peroxide 12% | 0.55; 0.50 | 4.1; 4.5 |
| Eosin + Urea Peroxide 6% | 0.55; 0.55 | 4.3; 4.0 |
| Eosin + KI 200 ppm + | 0.8; 0.95 | 3.1; 3.2 |
| Urea Peroxide 6% | | |
| Eosin + H₂O₂ 4.3% | 0.55; 0.55 | 4.04; 4.15 |
| Eosin + H₂O₂ 4.3% + KI 200 ppm | 1.00; 1.10 | 3.6; 3.0 |
| Eosin + KI 2000 ppm | 0.5 | 3.7 |
| Eosin + KI 2000 ppm + | 0.9 | 2.6 |
| Urea Peroxide 12% | | |

As can be seen, the addition of KI to a mixture of Eosin Y and peroxide significantly increased the singlet oxygen produced. In the case of 12% urea peroxide and Eosin Y, the singlet oxygen measure increases from 0.525 (average of 0.55 and 0.50) to 1.125 (average of 1.2 and 1.05) on addition of KI. In the case of 6% urea peroxide, the singlet oxygen measure increases from 0.55 to 0.875 on addition of KI. In the case of 4.3% hydrogen peroxide, the singlet oxygen measure increases from 0.55 to 1.05 on addition of KI. The singlet oxygen in the compositions with KI are shorter lived than in those compositions without KI.

An increase in the concentration of the KI from 200-2000 ppm did not increase the amount of singlet oxygen produced, suggesting that there may be a threshold concentration of KI above which the singlet oxygen production does not significantly increase.

Table 2 shows measurement of singlet oxygen in a diluted carbomer carrier gel. Again, as observed for the solutions of Table 1, the addition of KI (200 parts per million) to urea peroxide and eosin y increases the production of singlet oxygen.

TABLE 2
Singlet oxygen produced in diluted carbomer compositions with
eosin y, with and without 12% urea peroxide, and with and without
KI, according to embodiments of the present disclosure.

| | Singlet oxygen | Lifetime |
|---|---|---|
| Sample | measurement | (microseconds) |
| Eosin + Urea Peroxide 12% + carbomer | 0.5 | 4.2 |
| Eosin + carbomer | 0.5 | 3.9 |
| Eosin + KI + carbomer | 0.5 | 3.5 |
| Eosin + Urea Peroxide 12% + | 0.85 | 2.9 |
| KI + carbomer | | |

### Example 4—Concentration of KI

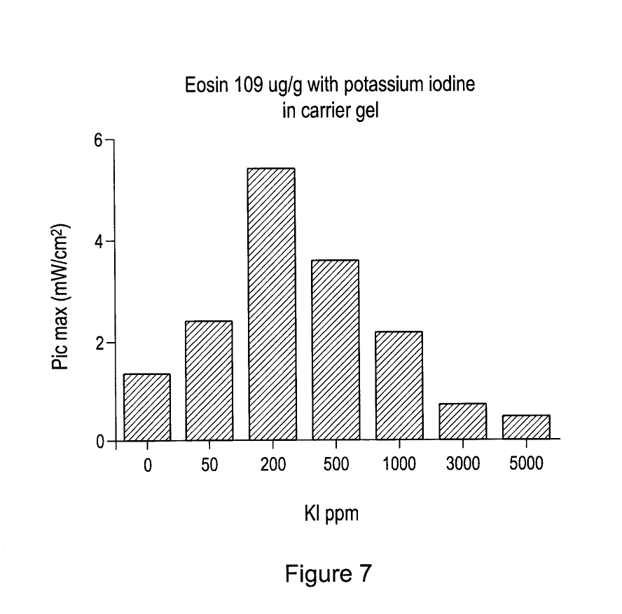

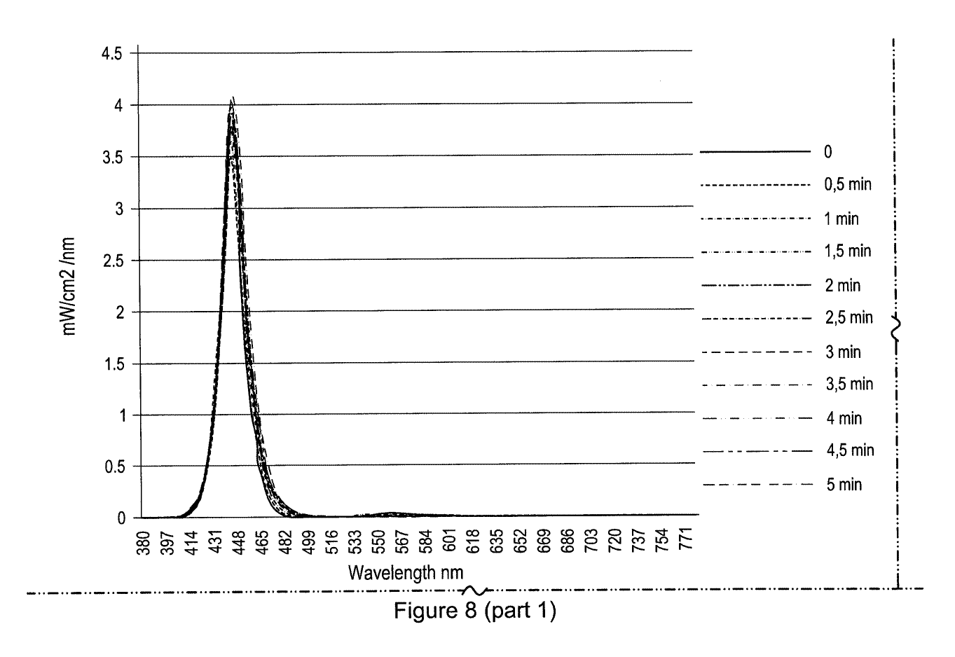

In this experiment, Eosin Y was added to a carbomer gel at a final concentration of 109 μg/g. Immediately after, KI was added to the gel. Seven separate samples were prepared, each with 0, 50, 200, 500, 1000, 3000, or 5000 ppm of KI. Following addition of KI, the gel was quickly mixed. The gel was then placed between two glass slides with a depth of 2 mm, and was illuminated by a blue lamp (5 cm distance) for 10 minutes. The fluorescence was measured and recorded by a spectrophotometer. The effect of KI in a composition comprising 109 ug/g Eosin Y and 12% urea peroxide is shown in Table 3 and FIG. 7.

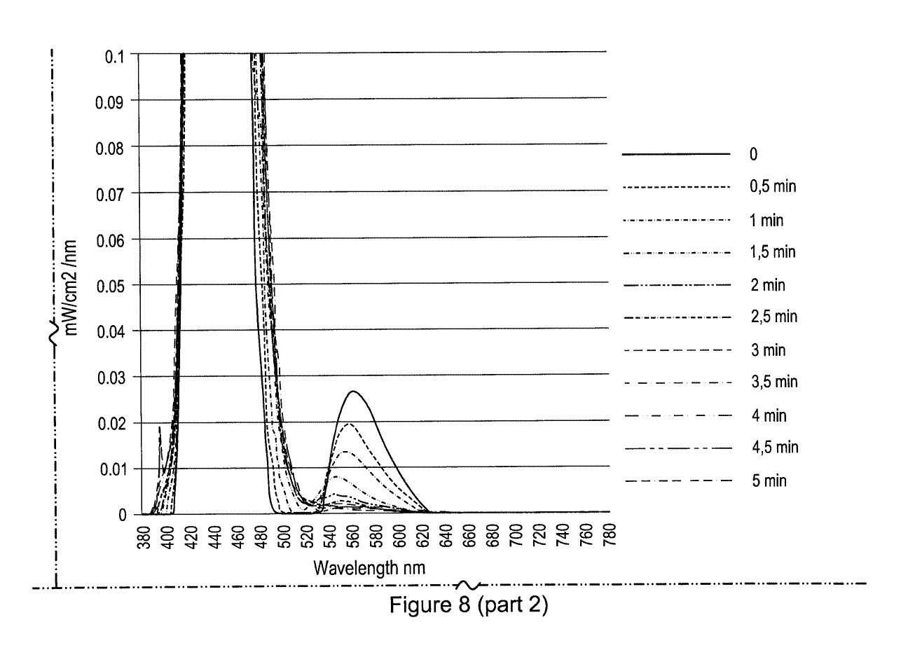

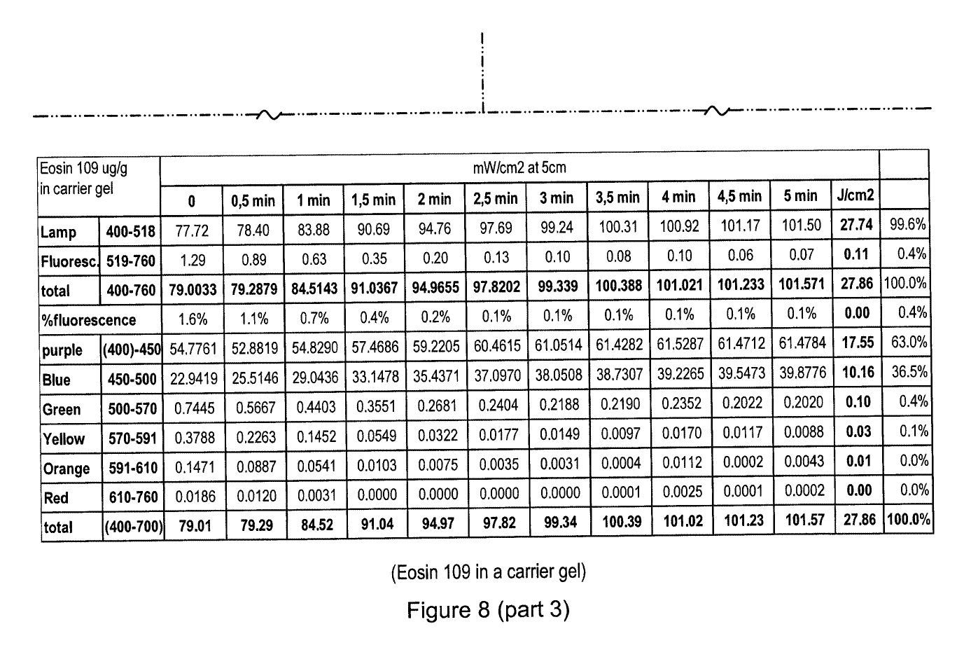

FIG. 8 shows the light spectrum recorded during exposure of a biophotonic composition comprising 109 ug/g Eosin Y, 12% urea peroxide and 0 ppm KI to blue light for 5 minutes.

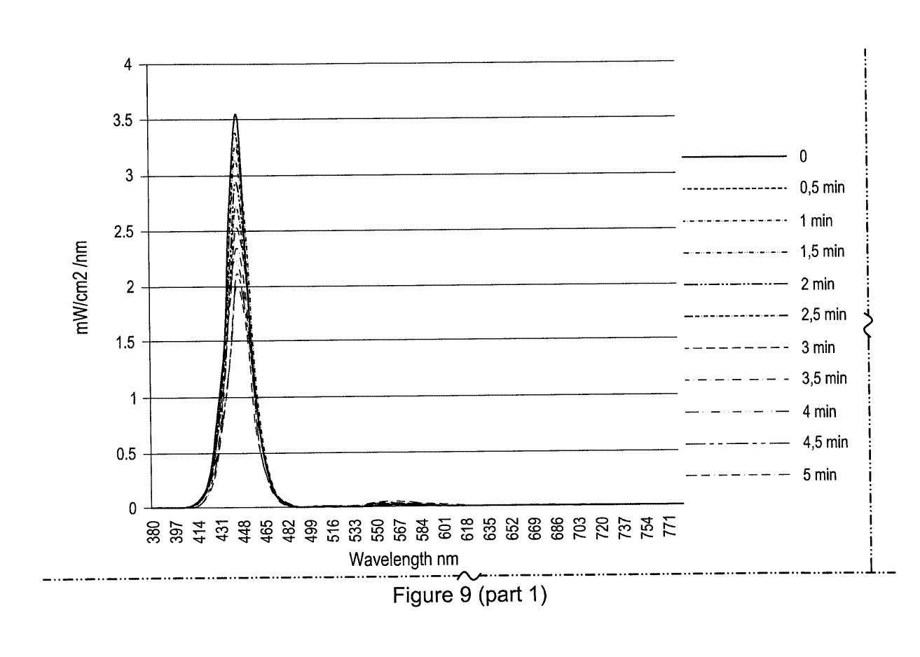

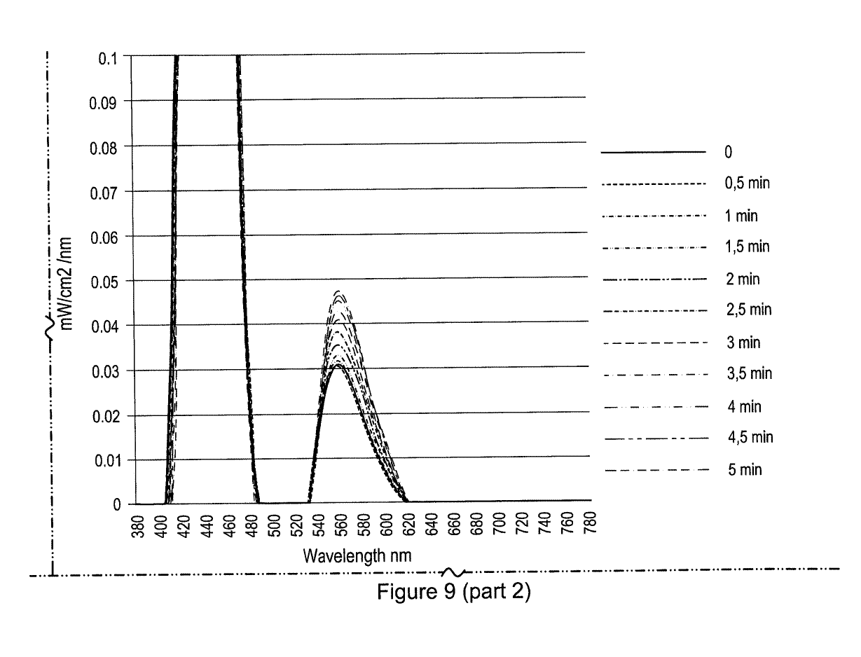

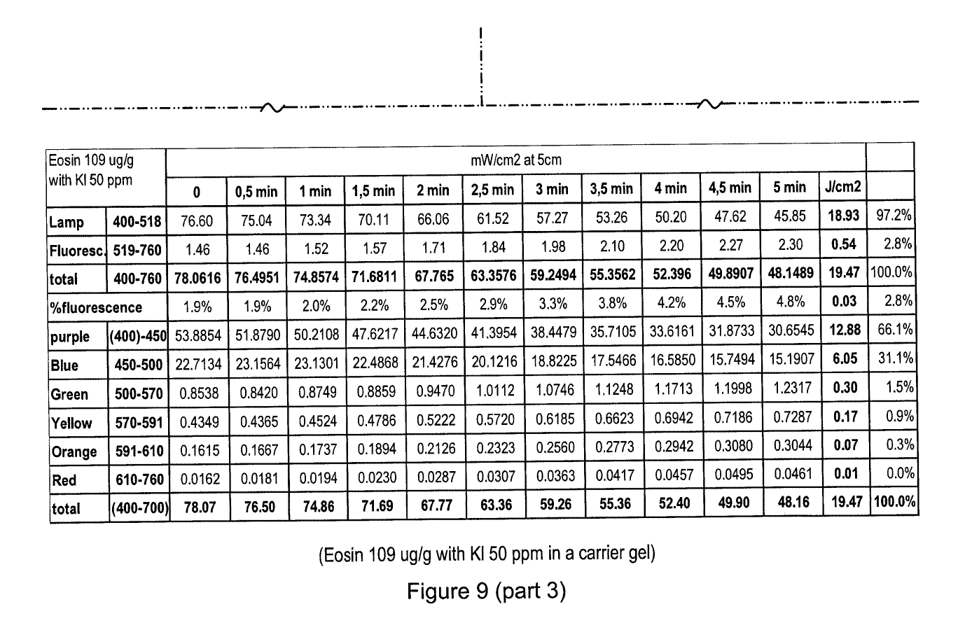

FIG. 9 shows the light spectrum recorded during exposure of a biophotonic composition comprising 109 ug/g Eosin Y, 12% urea peroxide and 50 ppm KI to blue light for 5 minutes.

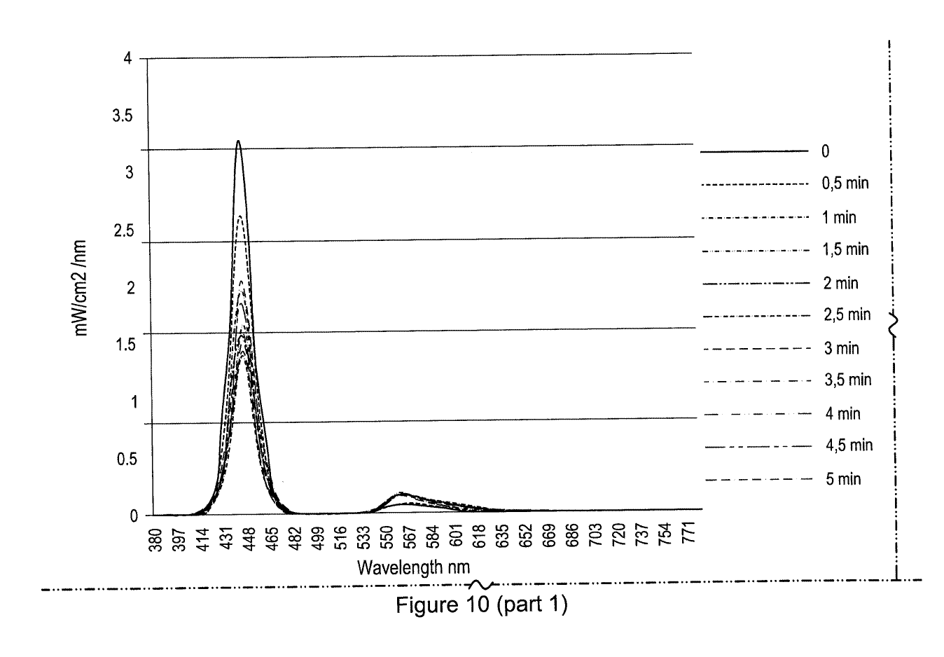

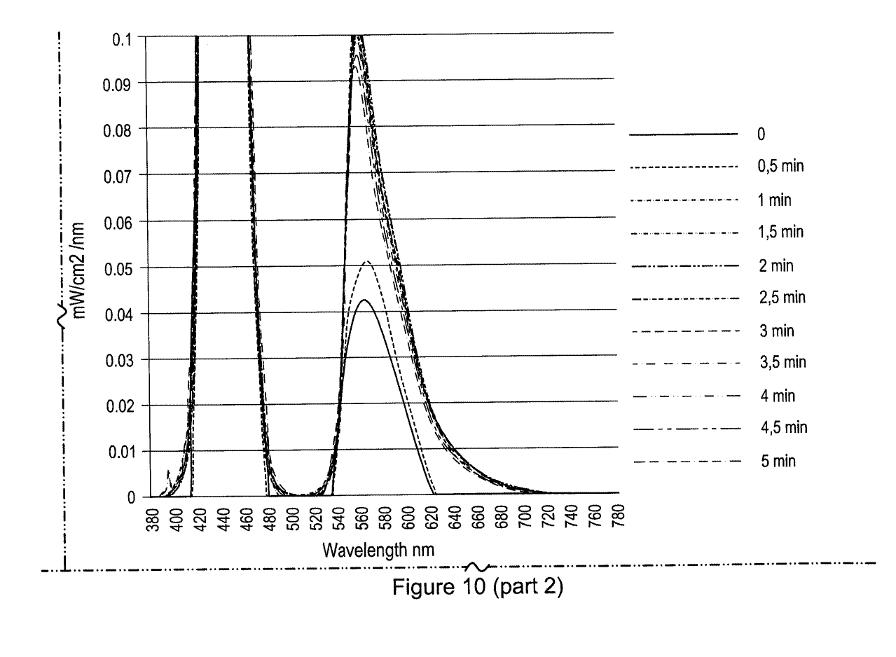

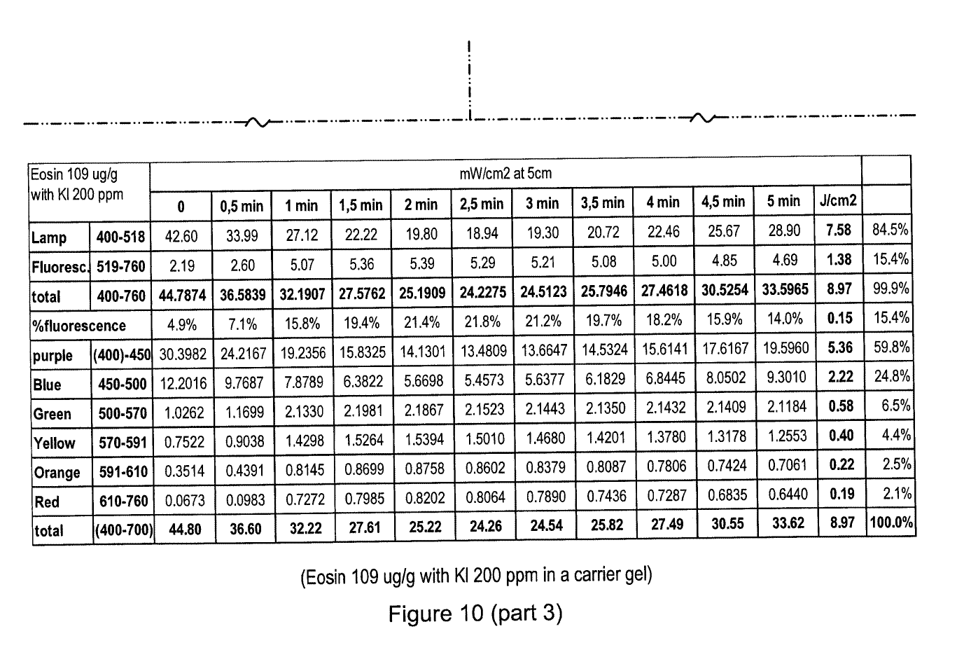

FIG. 10 shows the light spectrum recorded during exposure of a biophotonic composition comprising 109 ug/g Eosin Y, 12% urea peroxide and 200 ppm KI to blue light for 5 minutes.

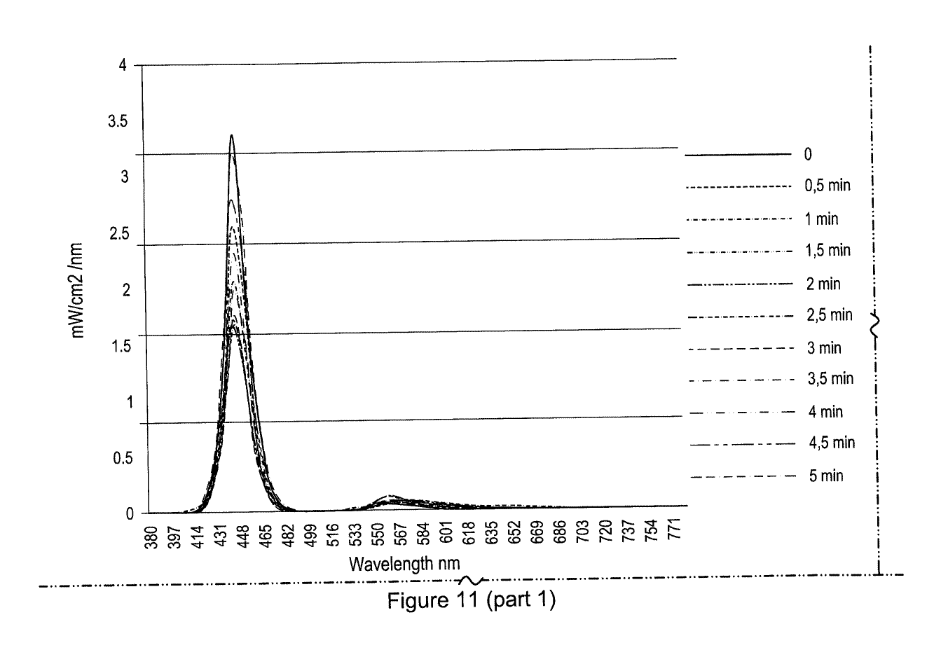

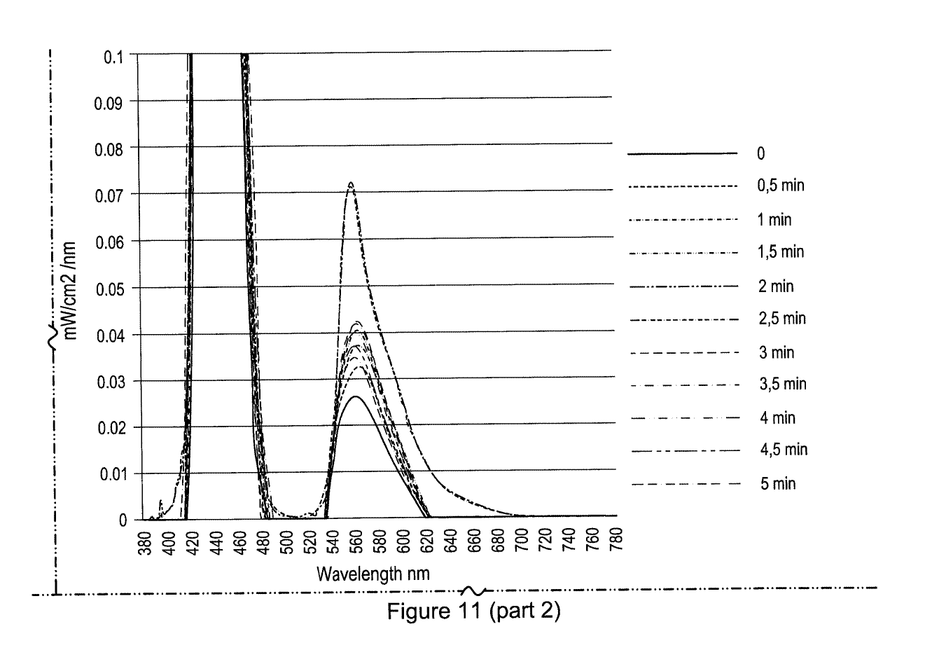

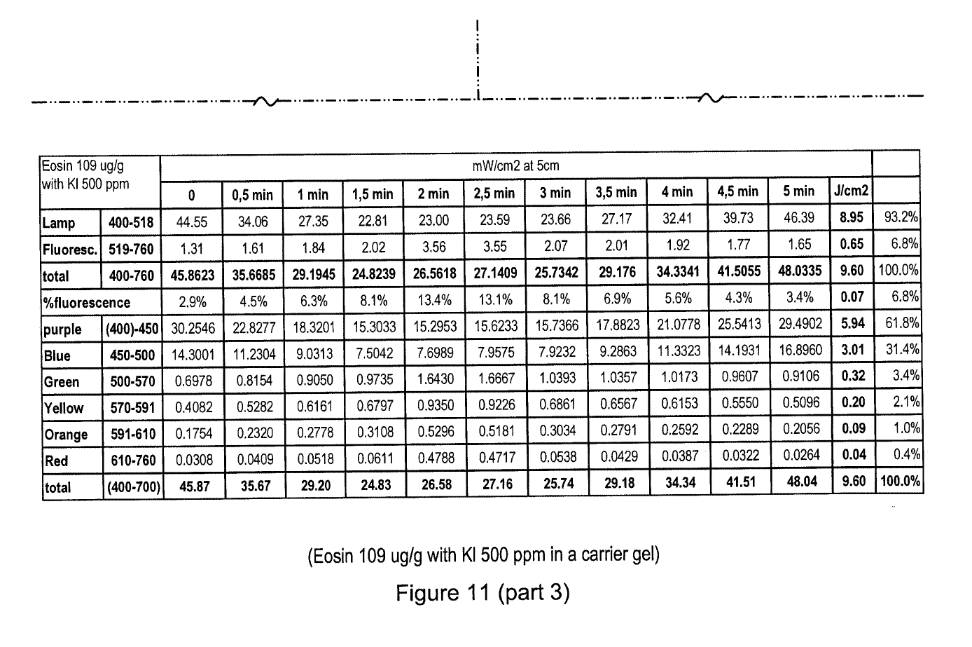

FIG. 11 shows the light spectrum recorded during exposure of a biophotonic composition comprising 109 ug/g Eosin Y, 12% urea peroxide and 500 ppm KI to blue light for 5 minutes.

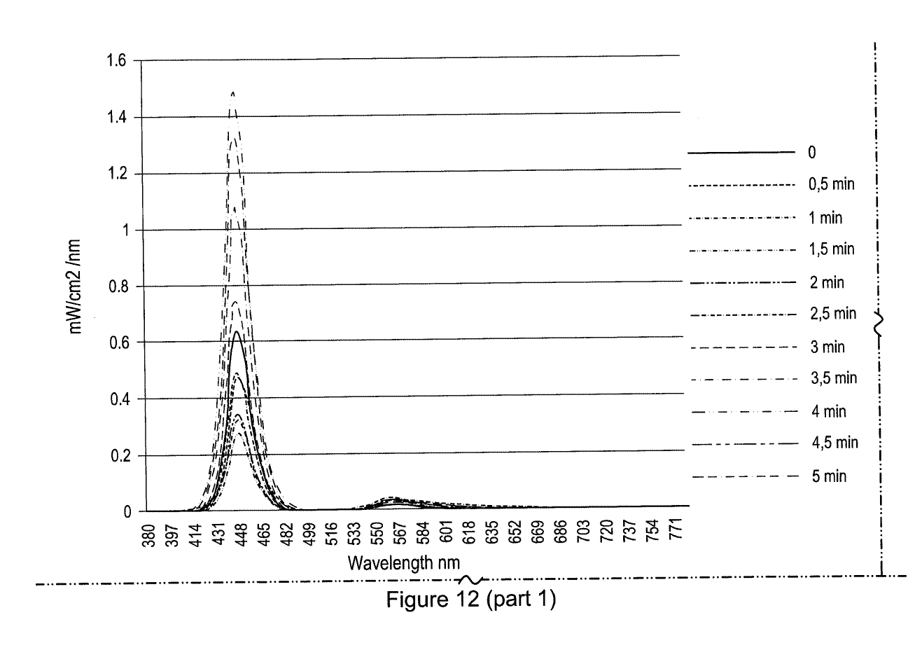

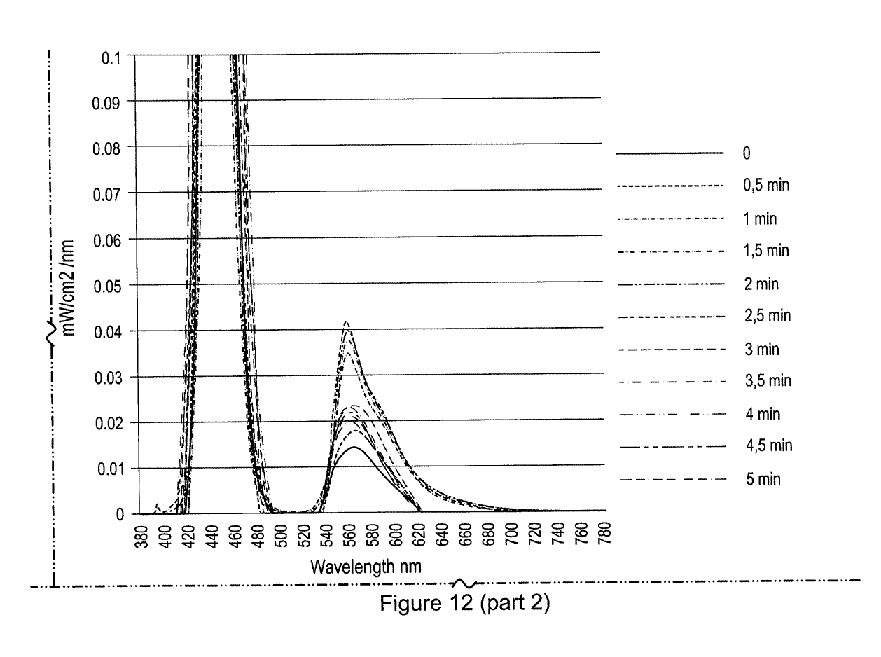

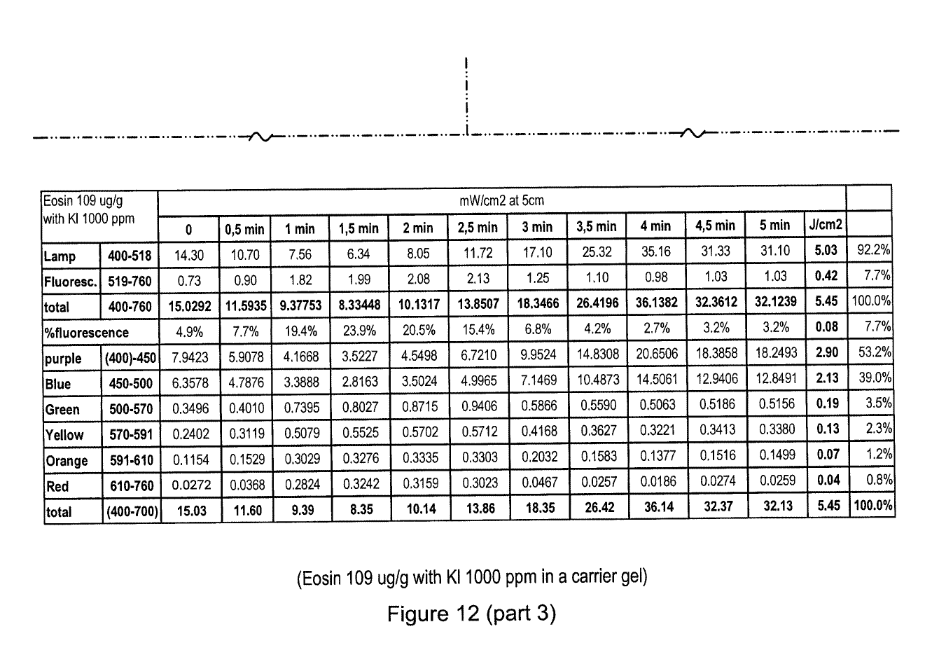

FIG. 12 shows the light spectrum recorded during exposure of a biophotonic composition comprising 109 ug/g Eosin Y, 12% urea peroxide and 1000 ppm KI to blue light for 5 minutes.

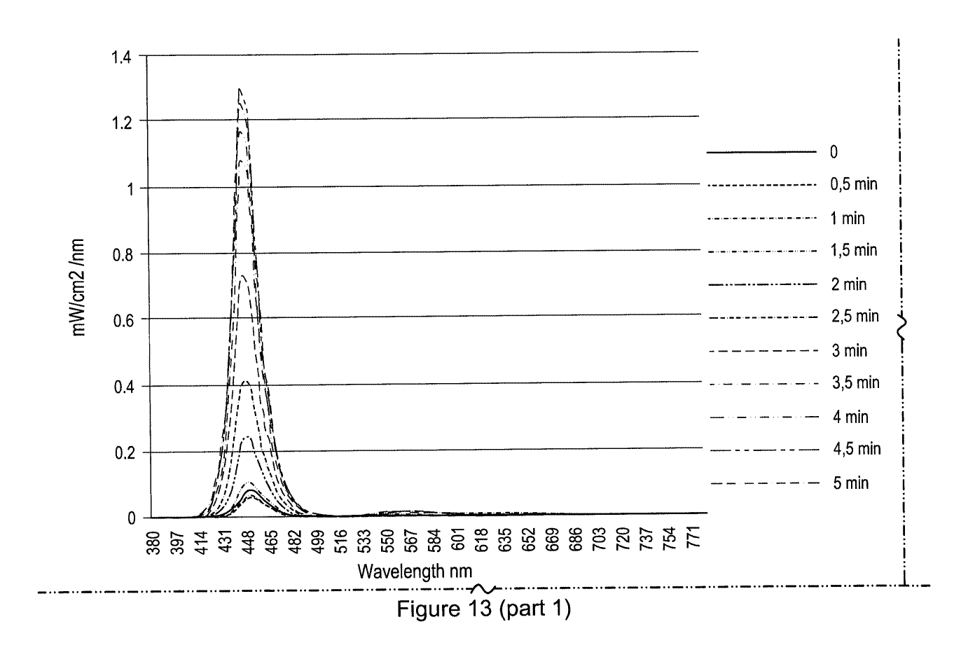

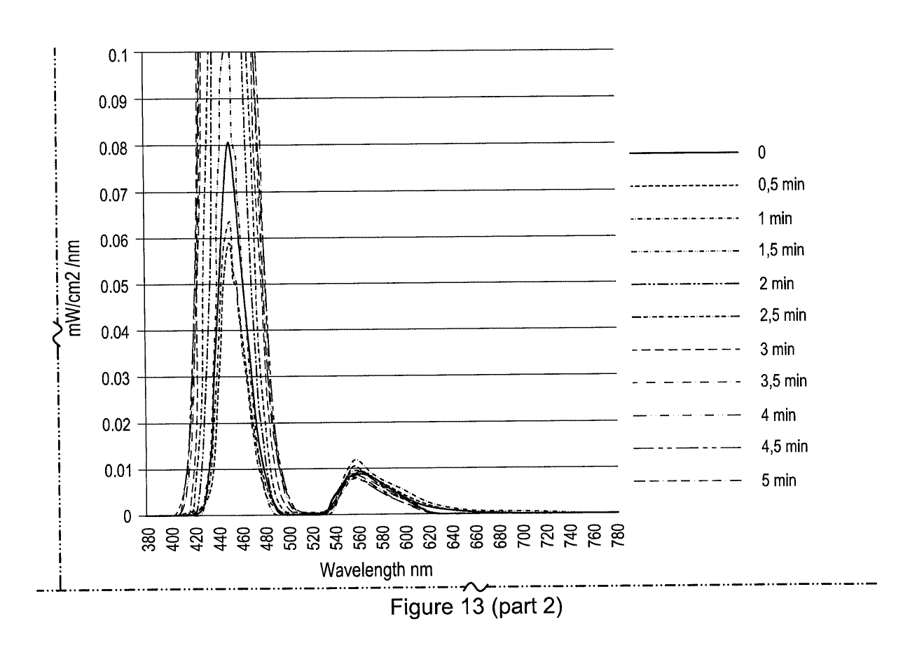

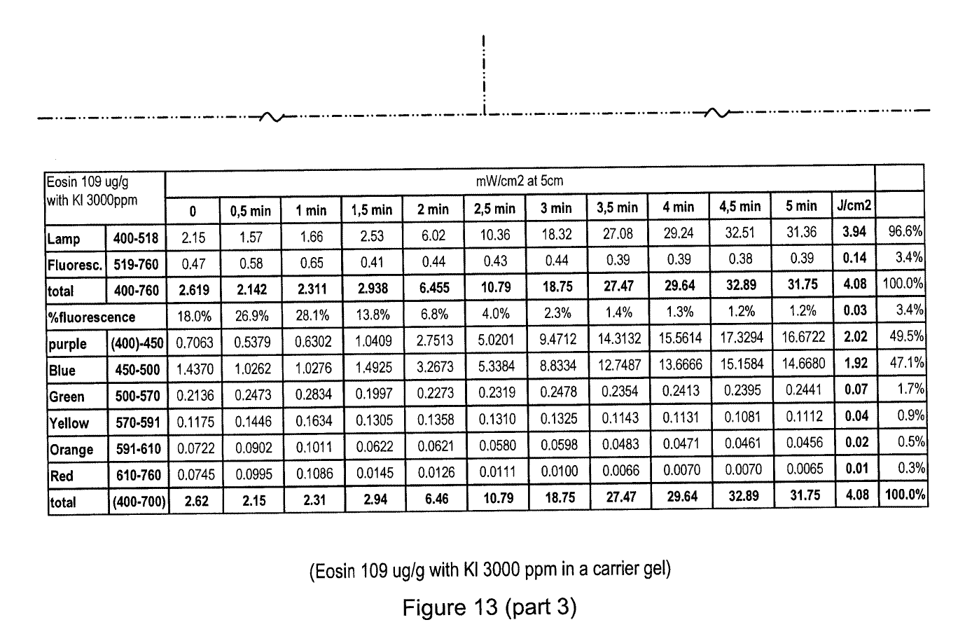

FIG. 13 shows the light spectrum recorded during exposure of a biophotonic composition comprising 109 ug/g Eosin Y, 12% urea peroxide and 3000 ppm KI to blue light for 5 minutes.

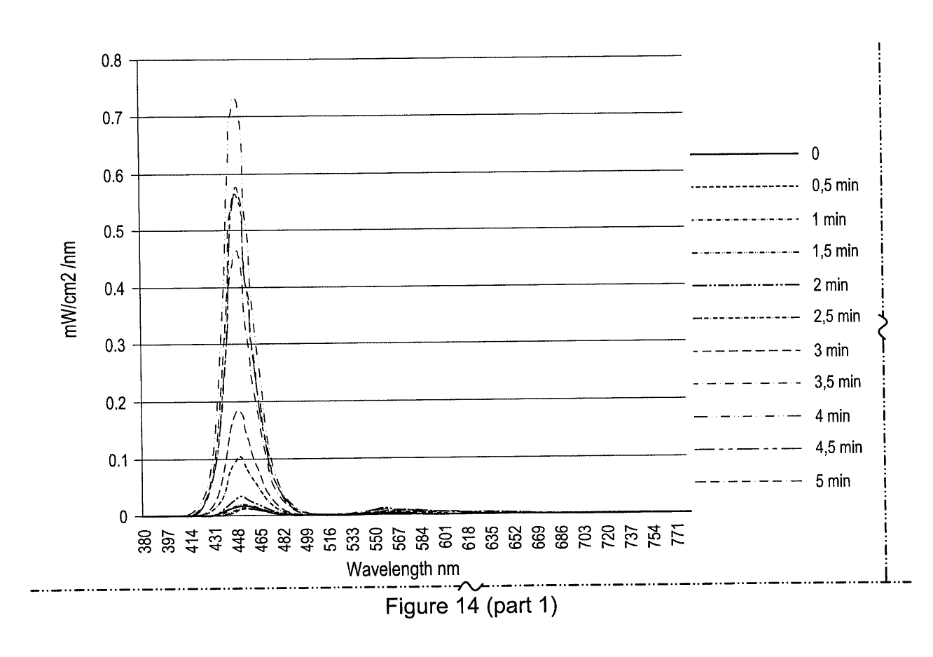

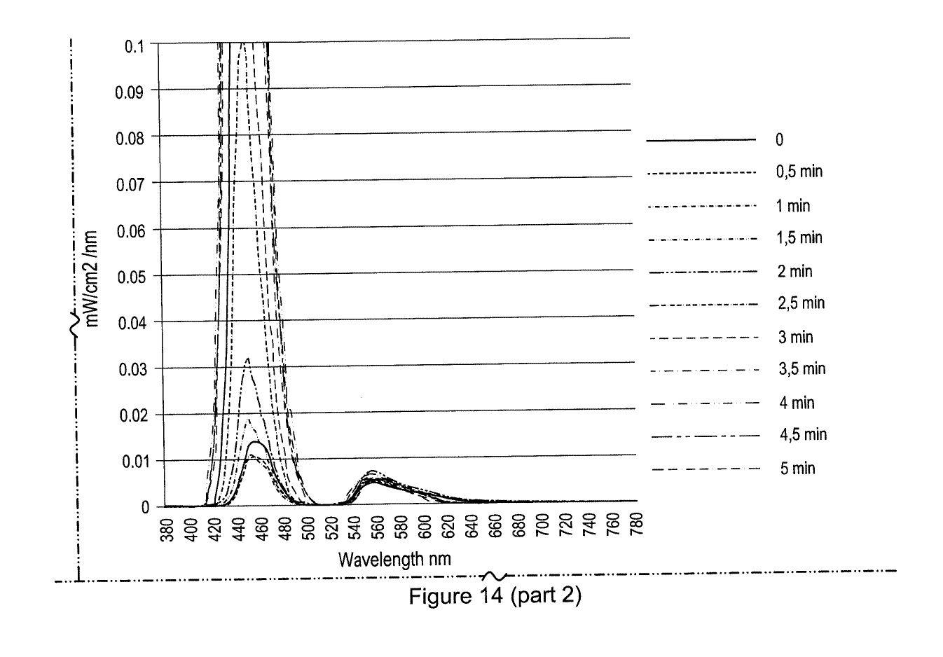

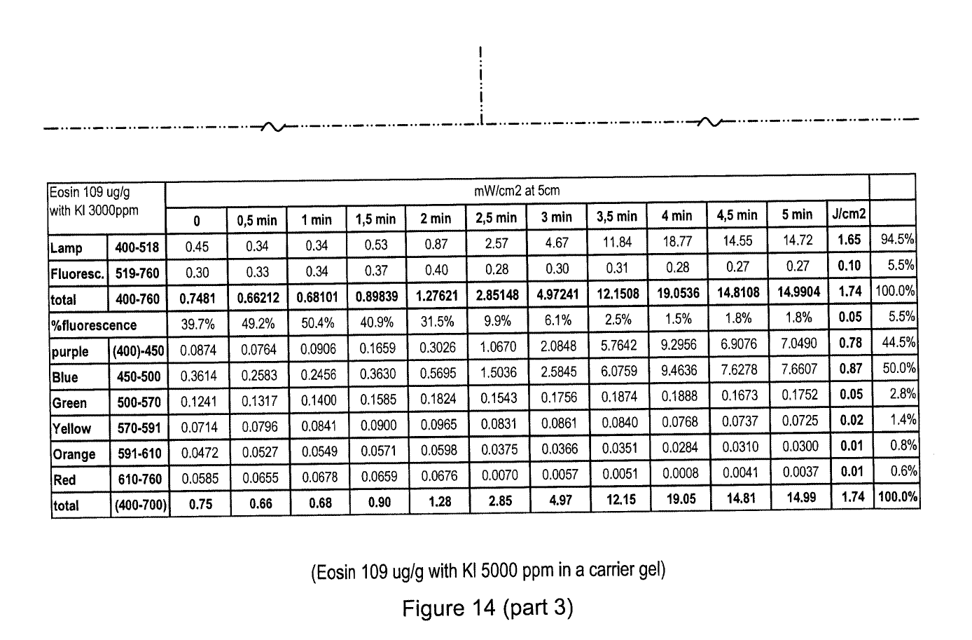

FIG. 14 shows the light spectrum recorded during exposure of a biophotonic composition comprising 109 ug/g Eosin Y, 12% urea peroxide and 5000 ppm KI to blue light for 5 minutes.

TABLE 3
Effect of KI concentration on the maximum emitted
fluorescence by eosin y when activated by a blue
light, and on the photobleaching time.

| | | Photobleaching |
|---|---|---|
| | Max peak fluorescence/ | time (min) |
| KI concentration/ppm | mW/cm² | (min 0.1 mW/cm²) |
| 0 | 1.29 (0 min) | 3 |
| 50 | 2.35 (6 min) | 48.5 |
| 200 | 5.39 (2 min) | 90 (0.28) |
| 500 | 3.56 (2 min) | 52 |
| 1000 | 2.13 (2.5 min) | 45 (0.56) |
| 3000 | 0.65 (1 min) | 15 (0.37) |
| 5000 | 0.40 (2 min) | 15 (0.24) |

It was found that the concentration of KI affected the amount of fluorescence emitted by Eosin Y in the presence of peroxide, as well as affecting the photobleaching time (lifespan) of the Eosin Y. The highest fluorescence level, and maximal photobleaching time, was observed at 200 ppm KI.

### Example 5—Singlet Oxygen Fluorescence

A Laser flash photolyser system composed of an excitation laser (Continuum Surelite SL IL-10) configured for the third harmonic at 355 nm and equipped with an Optical Parametric Oscillator (Continuum Surelite OPO Plus) tuned for 450 nm, and a Monochromator (Spectral Products CM-110 1/8m) as well as a Near Infrared photomultiplier system (Hamamatsu H10330-75) was used for singlet oxygen detection.

The gel samples were first loaded by aspiration in a 1 ml syringe without needle. The air trapped in the gel was removed by centrifugation of the syringe. For centrifugation, the syringe was capped and the piston was blocked in place. The assembly was centrifuged at 2000 for 5 minutes. The gel samples were then carefully transferred to a 1 nm quartz cuvette. The samples were excited by pulses of coherent light (typically 4×0.16 microsecond pulses). An excitation wavelength of 450 nm was used. Singlet oxygen fluorescence was measured at 1270 nm by single photon sensitive detector. The short-lived fluorescence data points were plotted and used to calculate the relative levels and average lifetime of singlet oxygen generated after the excitation pulses. Results are shown in Table 4.

TABLE 4
Singlet oxygen fluorescence and average lifetime in the indicated
compositions, according to embodiments of the present disclosure.

| | SINGLET | |
|---|---|---|
| | FLUO- | LIFETIME |
| SAMPLES | RESCENCE | (microseconds) |
| Eosin 2000X (218,000 μg) + | 0.55; 0.50 | 4.0; 4.0 |
| PBS | | |
| Eosin 2000X (218,000 μg) + | 0.60; 0.60 | 4.2; 4.0 |
| KI 2 ppm + H₂O | | |
| Eosin 2000X (218,000 μg) + | 0.6; 0.50 | 3.7; 4.1 |
| EDTA 100X (17,000 mg) + H₂O | | |
| Eosin 2000X (218,000 μg) + | 0.6; 0.45 | 3.6; 3.9 |
| EDTA 100X (17,000 mg) + KI 2 | | |
| ppm + H₂O | | |
| Eosin 2000X (218,000 μg) + | 0.55; 0.50 | 4.1; 4.5 |
| UP 12% + H₂O | | |
| Eosin 2000X (218,000 μg) + | 1.2; 1.05 | 3.25; 3.3 |
| KI 2 ppm + UP 12% + H₂O | | |
| Eosin 2000X (218,000 μg) + | 0.40; 0.50 | 4.4; 4.3 |
| UP 12% + EDTA 100X + H₂O | | |
| Eosin 2000X (218,000 μg) + | 1.20; 1.20 | 3.24; 3.2 |
| KI 2 ppm + UP 12% + EDTA | | |
| 100X (17,000 mg) + H₂O | | |
| Eosin 2000X (218,000 μg) + | 0.55; 0.55 | 4.04; 4.15 |
| H₂O₂ 4.3% + H₂O | | |
| Eosin 2000X (218,000 μg) + | 1.00; 1.10 | 3.6; 3.0 |
| H₂O₂ 4.3% + | | |
| KI 2 ppm + H₂O | | |
| Eosin + H₂O₂ 4.3% + | 0.50; 0.50 | 3.9; 4.0 |
| EDTA 100X (17,000 mg) + H₂O | | |
| Eosin + H₂O₂ 4.3% + | 1.20; 1.10 | 3.2; 3.3 |
| KI 2 ppm + EDTA 100X (17,000 mg) | | |
| Eosin 2000X (218,000 μg) + | 0.5 | 4.2 |
| UP12% + carbomer gel | | |
| Eosin 2000X (218,000 μg) + | 0.5 | 3.9 |
| carbomer gel | | |
| Eosin 2000X (218,000 μg) + | 0.5 | 3.5 |
| KI 2 ppm + carbomer gel | | |
| Eosin 2000X (218,000 μg) + | 0.85 | 2.9 |
| UP12% + KI 2 ppm + carbomer | | |
| gel | | |
| Eosin 2000X (218,000 μg) + | 1.1 | 3.3 |
| UP12% + KI 2 ppm + EDTA | | |
| 100X (17,000 mg) + carbomer gel | | |
| Eosin 2000X (218,000 μg) + | 0.5 | 4.1 |
| EDTA 100X (17,000 mg) + | | |
| carbomer gel | | |
| Carbomer gel only | 0 | 0 |
| Eosin 2000X (218,000 μg) + | 0.55 | 4 |
| carbomer gel | | |
| Eosin 2000X (218,000 μg) + | 0.6 | 4.2 |
| KI 2 ppm + carbomer gel | | |

*no blank subtracted for all samples

As can be seen, the addition of KI to the biophotonic compositions comprising an oxygen source in the form of urea peroxide significantly increased the singlet oxygen fluorescence.

### Example 6—Microfoam Production

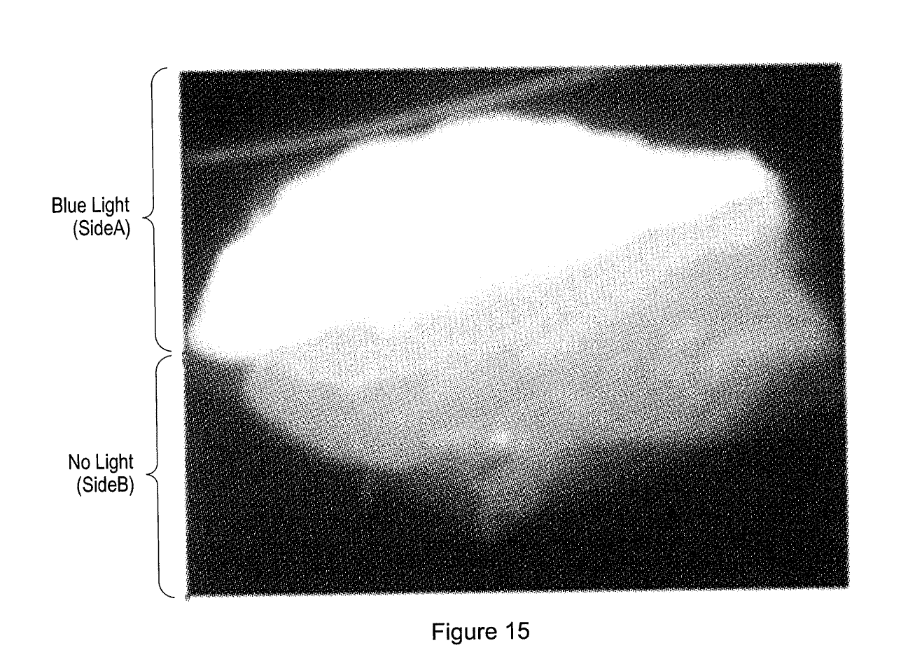

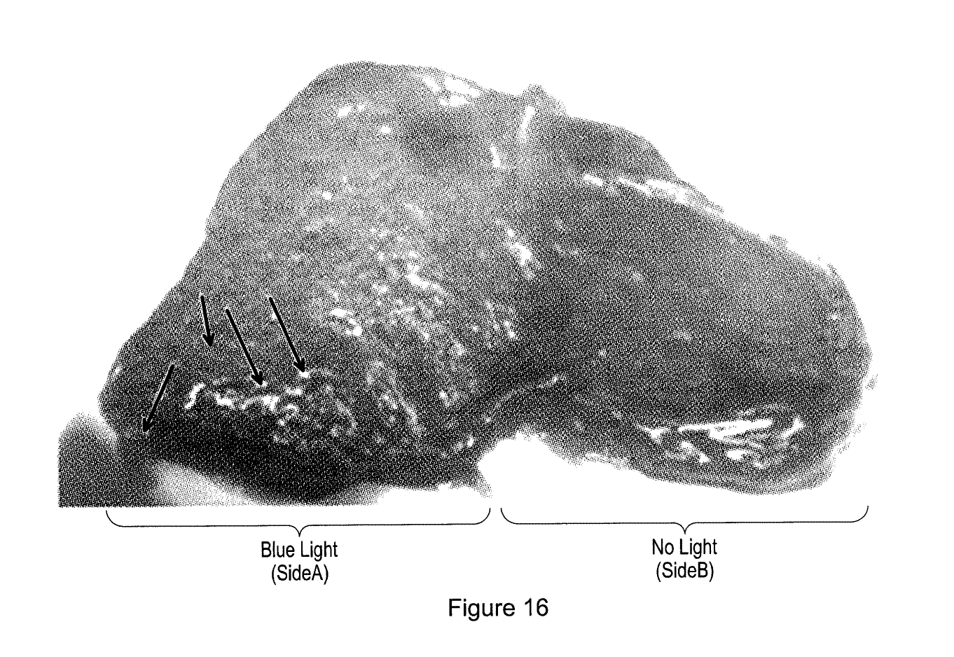

A carbomer gel comprising 109 ug/g Eosin Y, 12% urea peroxide and 200 ppm KI was prepared. Half of the gel was illuminated with blue light for a period of 5 minutes. The other half was not exposed to the blue light. As can be seen from FIG. 15, the illuminated half of the gel fluoresced (FIG. 15, Side A), while the non-illuminated side did not fluoresce (FIG. 15, Side B). Upon completion of the light exposure, the illuminated side of the gel was visually examined and a significant amount of microfoaming and swelling was evident (FIG. 16, Side A). Representative microbubbles are shown by the arrows in FIG. 16. The non-illuminated side of the gel lacked any microfoaming or swelling (FIG. 16, Side B). Of particular note, the illuminated side of the gel was not photobleached at the end of the 5 minute illumination period, retaining the same color as the non-illuminated side of the gel.

The microfoaming in the composition can have a debridement effect on a treatment area. For example, it can be used to clear dead cells from wounds, or to remove scaley skin in certain dermatological conditions. In fact, the American Medical Association has concluded that hydrogen peroxide may provide some mechanical benefit resulting from the effervescence loosening debris and necrotic tissue in the wound.

1-91. (canceled)

92. A biophotonic composition comprising:
at least one chromophore;
at least one halogen and/or halogen salt; and
a carrier.

93. (canceled)

94. The biophotonic composition of claim 92, further comprising an oxidant.

95. The biophotonic composition of claim 92, wherein said halogen and/or halogen salt is selected from the group consisting of KI, KCl, KBr, CsBr, MgBr₂, ZnBr₂, NaF, NaCl, NaBr, I₂, I₃⁻, Br₂, and Cl₂, and any combination thereof.

96-98. (canceled)

99. The biophotonic composition of claim 95, wherein said KI is at a concentration of from about 0.1 to about 100 ppm, or from about 0.1 to about 20 ppm, or from about 1 to 5000 ppm, or from about 100 to 300 ppm, or about 200 ppm.

100. The biophotonic composition of claim 94, wherein said oxidant is a peroxide or a peroxide precursor.

101. The biophotonic composition of claim 99, wherein said KI is at a concentration of about 200 ppm and the peroxide or the peroxide precursor is at a concentration of about 4.3%, or about 6%, or about 12%.

102-103. (canceled)

104. The biophotonic composition of claim 101, wherein said peroxide or peroxide precursor is selected from the group consisting of hydrogen peroxide, carbamide peroxide, benzoyl peroxide, peroxy acid, alkali metal peroxide, alkali metal percarbonate, peroxyacetic acid, alkali metal perborate, and methyl ethyl ketone peroxide.

105. (canceled)

106. The biophotonic composition of claim 101, wherein said peroxide or peroxide precursor is present in an amount of from about 0.01% to about 50% by weight of the final composition.

107-110. (canceled)

111. The biophotonic composition of claim 92, wherein said carrier is a synthetic polymer selected from the group consisting of vinyl polymers, polyoxyethylene-polyoxypropylene copolymers, poly(ethylene oxide), acrylamide polymers and derivatives or salts thereof.

112. The biophotonic composition of claim 111, wherein said carrier is a vinyl polymer selected from the group consisting of polyacrylic acid, polymethacrylic acid, polyvinyl pyrrolidone, and polyvinyl alcohol.

113-114. (canceled)

115. The biophotonic composition of claim 92, wherein said carrier is a polyacrylic acid polymer cross-linked with alkyl acrylate or allyl pentaerythritol and is present in an amount of from about 0.05% to about 5% by weight of the final composition, or from about 0.5% to about 2% by weight of the final composition.

116. The biophotonic composition of claim 92, wherein said carrier comprises a protein-based polymer selected from the group consisting of sodium hyaluronate, gelatin and collagen.

117. (canceled)

118. The biophotonic composition of claim 116, wherein said carrier is gelatin and is present in an amount of equal to or more than about 4% by weight of the final composition.

119. The biophotonic composition of claim 116, wherein said carrier is collagen and is present in an amount equal to or more than about 5% by weight of the final composition.

120. The biophotonic composition of claim 92, wherein said carrier comprises one or more polysaccharides selected from the group consisting of starch, chitosan, chitin, agar, alginates, xanthan, carrageenan, guar gum, gellan gum, pectin, and locust bean gum.

121. (canceled)

122. The biophotonic composition of claim 92, wherein said carrier comprises at least one glycol selected from the group consisting of ethylene glycol and propylene glycol.

123-126. (canceled)

127. The biophotonic composition of claim 92, wherein said chromophore is a xanthene dye.

128. The biophotonic composition of claim 127, wherein said xanthene dye is selected from the group consisting of Eosin, Erythrosin B, Fluorescein, Rose Bengal, and Phloxin B.

129. The biophotonic composition of claim 92, wherein said chromophore is present in an amount of from about 0.0001% to about 40% by weight of the total composition, or from about 0.0001% to about 2% by weight of the total composition.

130. The biophotonic composition of claim 92, wherein the composition further comprises a second chromophore.

131. The biophotonic composition of claim 130, wherein the first chromophore has an emission spectrum that overlaps at least 20% with an absorption spectrum of the second chromophore.

132. (canceled)

133. The biophotonic composition of claim 130, wherein said second chromophore is selected from the group consisting of Fluorescein, Phloxine B and Erythrosine B.

134. (canceled)

135. The biophotonic composition of claim 130, wherein said second chromophore is present in an amount of from about 0.0001% to about 40% by weight of the total composition, or from about 0.0001% to about 2% by weight of the total composition.

136. The biophotonic composition of claim 130, further comprising a third chromophore, wherein the third chromophore is a chlorophyll or saffron.

137. The biophotonic composition of claim 136, wherein said third chromophore is present in an amount of from about 0.0001% to about 40% by weight of the total composition, or from about 0.0001% to about 2% by weight of the total composition.

138-143. (canceled)

144. A method for biophotonic skin treatment, comprising:
applying a biophotonic composition to a target skin tissue, wherein the biophotonic composition comprises:
at least one chromophore; and
at least one halogen and/or halogen salt selected from the group consisting of KI, KCl, KBr, CsBr, MgBr₂, ZnBr₂, NaF, NaCl, NaBr, I₂, I₃⁻, Br₂, Cl₂, and any combination thereof; and
illuminating said biophotonic composition with light having a wavelength that overlaps with an absorption spectrum of the chromophore.

145. (canceled)

146. A method for promoting wound healing comprising:
applying a biophotonic composition over or within a wound, wherein the biophotonic composition comprises:
at least one chromophore; and
at least one halogen and/or halogen salt selected from the group consisting of KI, KCl, KBr, CsBr, MgBr₂, ZnBr₂, NaF, NaCl, NaBr, I₂, I₃⁻, Br₂, Cl₂, and any combination thereof; and
illuminating said biophotonic composition with light having a wavelength that overlaps with an absorption spectrum of the chromophore.

147-149. (canceled)

150. A kit comprising:
a first component comprising at least one chromophore; and
a second component comprising at least one halogen and/or halogen salt selected from the group consisting of KI, KCl, KBr, CsBr, MgBr₂, ZnBr₂, NaF, NaCl, NaBr, I₂, I₃⁻, Br₂, Cl₂, and any combination thereof.

151-159. (canceled)

160. The method of claim 144, wherein the skin treatment comprises treating a skin disorder selected from the group consisting of acne, eczema, psoriasis, and dermatitis.

161. The method of claim 146, wherein the method comprises preventing or treating scarring.
